(19) United States
(12) Patent Application Publication
(10) Pub. No.: US 2025/0243188 A1
(43) Pub. Date: Jul. 31, 2025

(54) METHIONINE ADENOSYLTRANSERASE 2A INHIBITORS

(71) Applicant: IDEAYA BIOSCIENCES, INC., South San Francisco, CA (US)

(72) Inventors: Melissa FLEURY, South San Francisco, CA (US); Paul A. BARSANTI, South San Francisco, CA (US); Ethan DeNardo MCSPADDEN, South San Francisco, CA (US)

(21) Appl. No.: 18/854,017

(22) PCT Filed: Apr. 7, 2023
(86) PCT No.: PCT/US2023/065540

§ 371 Date: Oct. 3, 2024

Related U.S. Application Data
(60) Provisional application No. 63/328,921, filed on Apr. 8, 2022.

(51) Int. Cl. C07D 409/14; A61K 31/517; C07D 239/96; C07D 403/14; C07D 417/14

(52) U.S. Cl. CPC C07D 409/14; A61K 31/517; C07D 239/96; C07D 403/14; C07D 417/14

(57) ABSTRACT

Disclosed herein are compounds of Formula (I): or a pharmaceutically acceptable salt thereof, wherein A and A′ are each independently selected from the group consisting of: (a1), (a2), (a3), (4) and (a5) and B, B′, X and Y have the meanings provided herein, that are methionine adenosyltransferase 2A (MAT2A) inhibitors. Also disclosed are pharmaceutical compositions comprising such compounds and methods of treating diseases treatable by inhibition of MAT2A such as cancer.

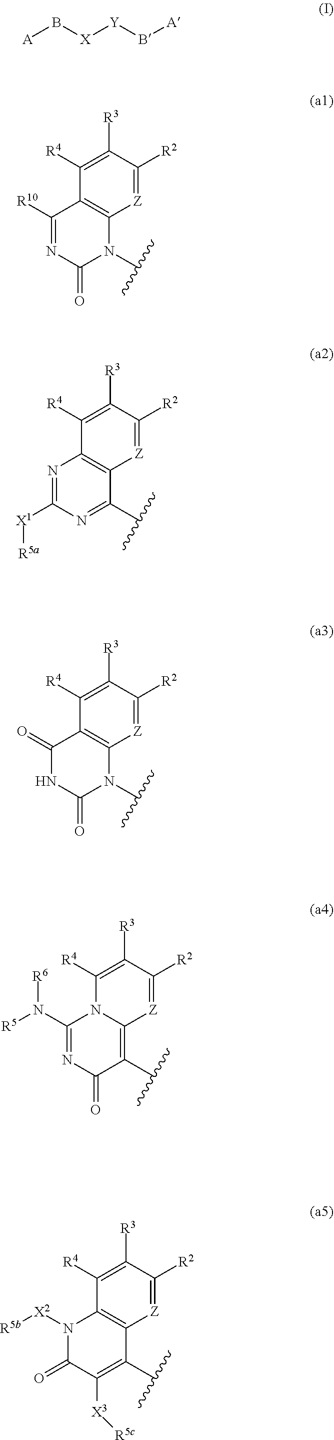

### CROSS-REFERENCES TO RELATED APPLICATIONS

This application claims the benefit under 35 U.S.C. 119(e) of U.S. Provisional Application No. 63/328,921, filed on Apr. 8, 2022, the contents of which is hereby incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cancer is a leading cause of death throughout the world. A limitation of prevailing therapeutic approaches, e.g. chemotherapy and immunotherapy is that their cytotoxic effects are not restricted to cancer cells and adverse side effects can occur within normal tissues. Consequently, novel strategies are needed to better target cancer cells.

Synthetic lethality arises when a combination of deficiencies in the expression of two or more genes leads to cell death, whereas a deficiency in only one of these genes does not. The concept of synthetic lethality originates from studies in drosophila model systems in which a combination of mutations in two or more separate genes leads to cell death (in contrast to viability, which occurs when only one of the genes is mutated or deleted). More recently, a multitude of studies have explored maladaptive genetic changes in cancer cells that render them vulnerable to synthetic-lethality approaches. These tumor-specific genetic defects lead to the use of targeted agents that induce the death of tumor cells while sparing normal cells.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA and proteins. One methylase that utilizes SAM as a methyl donor, is protein arginine N-methyltransferase 5 (PRMT5). While SAM is required for PRMT5 activity, PRMT5 is competitively inhibited by 5′methylthioadenosine (MTA). Since MTA is part of the methionine salvage pathway, cellular MTA levels stay low in a process initiated by methylthioadenosine phosphorylase (MTAP).

MTAP is in a locus on chromosome 9 that is often deleted in cells of patients with cancers from several tissues of origin including central nervous system, pancreas, esophageal, bladder and lung (cBioPortal database). Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted cells compare to MTAP WT cells (see McDonald et. al. 2017 Cell 170, 577-592). Furthermore, inducible knockdown of MAT2A protein decreased tumor growth in vivo (see Marjon et. al., 2016 Cell Reports 15(3), 574-587). These results indicate that MAT2A inhibitors may provide a novel therapy for cancer patients including those with MTAP-deleted tumors.

### SUMMARY

Disclosed herein are certain quinolinone derivatives that are methionine adenosyltransferase 2A (MAT2A) inhibitors. Also disclosed are pharmaceutical compositions comprising such compounds and methods of treating diseases treatable by inhibition of MAT2A such as cancer, including cancers characterized by reduced or absence of methylthioadenosine phosphorylase (MTAP) activity.

In a first aspect, provided is a compound of Formula (I):

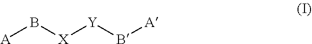

or a pharmaceutically acceptable salt thereof, wherein
A and A′ are each independently selected from the group consisting of:

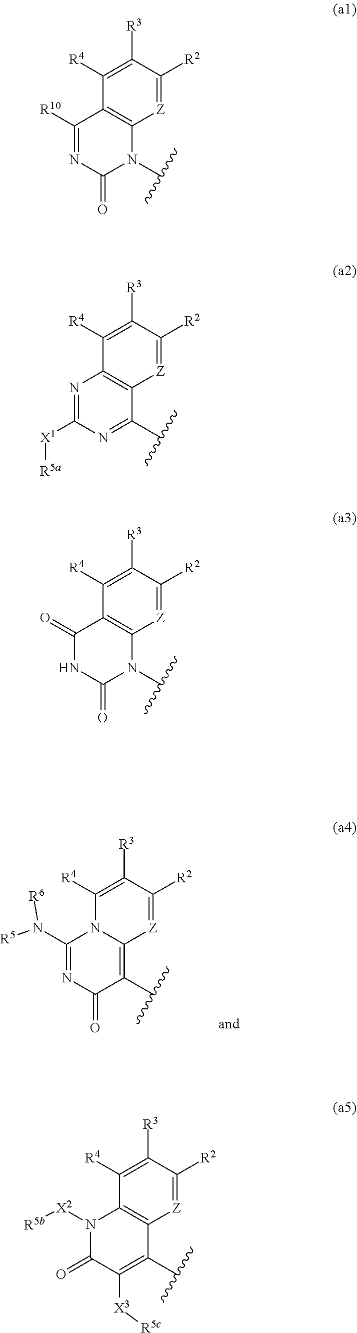

wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R⁸ is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6-to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁-6 haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

In a second aspect, provided is a pharmaceutical composition comprising a compound of Formula (1), a subembodinent described herein, or a phamaceutically acceptable satl thereof and at least one pharmaceutically acceptable excipient.

In a third aspect, provided is a method for treating a disease mediated by MAT2A in a patient comprising administering to the patient a therapeutically effective amount of a compound of Formula (I), a subembodiment described herein, or a pharmaceutically acceptable salt thereof. In first embodiment of the third aspect, the patient is in recognized need of such treatment. In second embodiment of the third aspect and first embodiment contained therein, the compound of Formula (I), a subembodiment described herein, or a pharmaceutically acceptable salt thereof is administered in a pharmaceutical composition. In a third embodiment of the third aspect and first and second embodiments contained therein, the disease is mediated by overexpression of MAT2A. In fourth embodiment of the third aspect and first, second, and third embodiments contained therein, the disease is cancer.

In a fourth aspect, provided is a method of treating a MTAP null cancer in a patient comprising administering to the patient a therapeutically effective amount of a compound of Formula (I), a subembodiment described herein, or a pharmaceutically acceptable salt thereof. In first embodiment of the fourth aspect, the patient is in recognized need of such treatment. In second embodiment of the fourth aspect and first embodiment contained therein, the compound of Formula (I), a subembodiment described herein, or a pharmaceutically acceptable salt thereof is administered in a pharmaceutical composition.

In a fifth aspect, provided is a method for inhibiting the synthesis of S-adenosyl methionine (SAM) from methionine and ATP by MAT2A in a cell comprising contacting the cell with an effective amount of a compound of Formula (I), a subembodiment described herein, or a pharmaceutically acceptable salt thereof.

In a sixth aspect, provided is a method for treating a cancer in a patient, wherein the cancer is characterized by a reduction or absence of methylthioadenosine phosphorylase (MTAP) gene expression, the absence of the MTAP gene, reduced function of MTAP protein, reduced level or absence of MTAP protein, MTA accumulation, or combination thereof, comprising administering to the subject a therapeutically effective amount of a compound of Formula (I), a subembodiment described herein, or a pharmaceutically acceptable salt thereof, and optionally in a pharmaceutical composition.

In a seventh aspect, provided is a compound of Formula (I) a subembodiment described herein, or a pharmaceutically acceptable salt thereof for inhibiting the synthesis of S-adenosyl methionine (SAM) from methionine and ATP by MAT2A in a cell.

In an eighth aspect, provided is a compound of Formula (I), a subembodiment described herein, or a pharmaceutically acceptable salt thereof, or a pharmaceutical composition as defined herein, for use in the treatment of a disease in a patient, wherein the disease is mediated by the overexpression of MAT2A.

In a ninth aspect, provided is a compound of Formula (I), a subembodiment described herein, or a pharmaceutically acceptable salt thereof, or a pharmaceutical composition as defined herein, for use in the treatment a cancer in a patient, wherein the cancer is characterized by a reduction or absence of methylthioadenosine phosphorylase (MTAP) gene expression, the absence of the MTAP gene, reduced function of MTAP protein, reduced level or absence of MTAP protein, MTA accumulation, or combination thereof.

In a tenth aspect, provided is a provided a compound of Formula (I), a subembodiment described herein, or a pharmaceutically acceptable salt thereof, or a pharmaceutical composition as defined herein, in the manufacture of a medicament for use in the treatment of cancer. Suitably, the medicament is for use in the treatment of human cancers.

### DETAILED DESCRIPTION

Before the present invention is further described, it is to be understood that the invention is not limited to the particular embodiments set forth herein, and it is also to be understood that the terminology used herein is for the purpose of describing particular embodiments only, and is not intended to be limiting.

The singular forms “a,” “an,” and “the” as used herein and in the appended claims include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or tert-butoxy, and the like.

“Aryl” means a monovalent monocyclic or bicyclic aromatic hydrocarbon radical of 6 to 10 ring atoms e.g., phenyl or naphthyl.

“Cycloalkyl” means a monocyclic monovalent hydrocarbon radical of three to eight carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heterocycloalkyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocycloalkyl ring can optionally be replaced by a —CO— group. More specifically the term heterocycloalkyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocycloalkyl ring is unsaturated it can contain one or two ring double bonds provided that the ring is not aromatic.

“Oxo,” as used herein, alone or in combination, refers to ═(O).

“Pharmaceutically acceptable salts” as used herein is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The present disclosure also includes protected derivatives of compounds of the present disclosure. For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes prodrugs of the compound of Formula (I), a subembodiment described herein, or a pharmaceutically acceptable salt thereof. Prodrugs of the compounds described herein are those compounds that readily undergo chemical changes under physiological conditions to provide the compounds of the present invention. An example, without limitation, of a prodrug would be a compound which is administered as an ester (the “prodrug”), but then is metabolically hydrolyzed to the carboxylic acid, the active entity. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of Formula (I) or a subembodiment described herein can exist in unsolvated forms as well as solvated forms, including hydrated forms. In general, the solvated forms are equivalent to unsolvated forms and are intended to be encompassed within the scope of the present invention. Certain compounds of Formula (I) or a subembodiment described herein may exist in multiple crystalline or amorphous forms. In general, all physical forms are equivalent for the uses contemplated by the present disclosure and are intended to be within the scope of the present disclosure.

Certain compounds of Formula (I) or a subembodiment described herein possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of Formula (I) or a subembodiment described herein may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (I), a subembodiment described herein (including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” and as used herein includes all mammals including humans. Examples of patients include humans, livestock such as cows, goats, sheep, pigs, and rabbits, and companion animals such as dogs, cats, rabbits, and horses. Preferably, the patient is a human.

“In need of treatment” as used herein refers to a judgment made by a physician or other caregiver that a subject requires or will benefit from treatment. This judgment is made based on a variety of factors that are in the realm of the physician's or caregiver's expertise.

“Administration”, “administer” and the like, as they apply to, for example, a patient, cell, tissue, organ, or biological fluid, refer to contact of, for example, a compound of Formula (I), a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Therapeutically effective amount” as used herein means the amount of a compound of Formula (I) or a subembodiment described herein and/or a pharmaceutically acceptable salt thereof that, when administered to a patient for treating a disease either alone or as part of a pharmaceutical composition and either in a single dose or as part of a series of doses, is sufficient to affect such treatment for the disease. The “therapeutically effective amount” will vary depending on the compound, the disease and its severity and the age, weight, etc., of the mammal to be treated. The therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I) or a subembodiment described herein (or, e.g., a metabolite thereof) at a particular time post-administration may be indicative of whether a therapeutically effective amount has been used.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

“Inhibiting”, “reducing,” or any variation of these terms in relation of MAT2A, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of MAT2A activity compared to its normal activity.

### Compounds of Formula (I)

In some aspects, provided herein are compounds having the Formula (I)

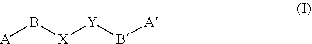

or a pharmaceutically acceptable salt thereof, wherein
A and A′ are each independently selected from the group consisting of:

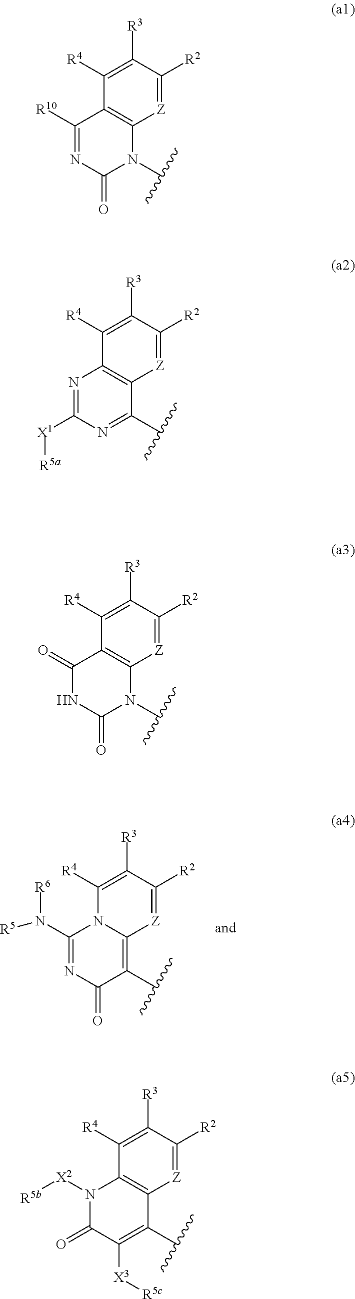

wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

In some embodiments, the compounds described herein are represented by Formula (I), above, wherein:
A and A′ are each independently selected from the group consisting of:

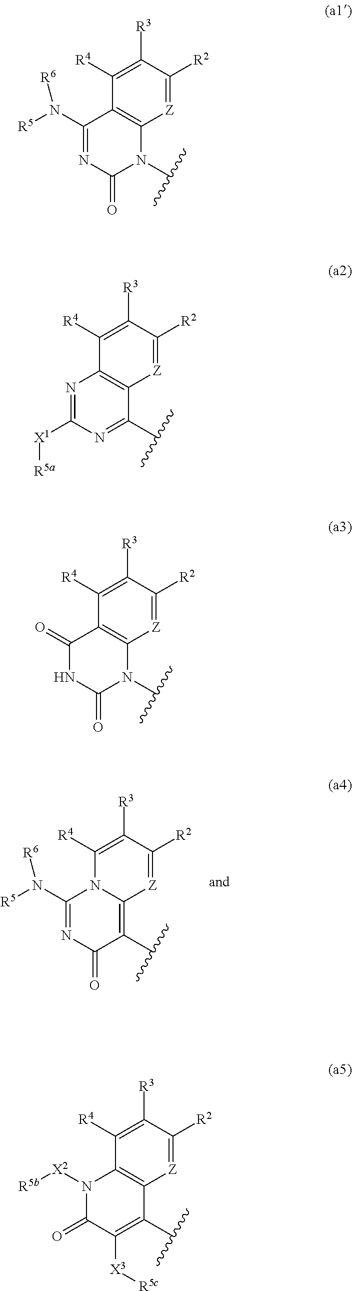

wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(CH₃), O and S;
Y is selected from the group consisting of CH₂, C(O), NH, N(CH₃), O and S;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R⁵ and R⁶ are each independently selected from the group consisting of H, C₁₋₆ alkyl, C₁₋₆ haloalkyl, and C₃₋₆ cycloalkyl;
each R⁷ is independently selected from the group consisting of halogen, cyano, C₁₋₃ alkyl, and C₁₋₃ haloalkyl;
X¹ is selected from the group consisting of a bond, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond and C₁₋₄ alkylene;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁-6 haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{Y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

In some embodiments, the compounds described herein are represented by Formula (I), or a pharmaceutically acceptable salt thereof as described in the embodiments above, wherein each A and A′ has the formula:

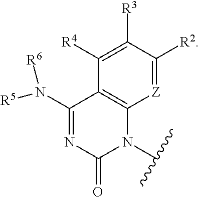

In some embodiments, the compounds described herein are represented by Formula (I), or a pharmaceutically acceptable salt thereof as described in the embodiments above, wherein each A and A′ has the formula:

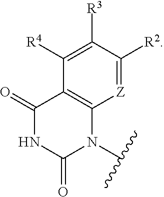

In some embodiments, the compounds described herein are represented by Formula (I), or a pharmaceutically acceptable salt thereof as described in the embodiments above, wherein only one of A and A′ has the formula:

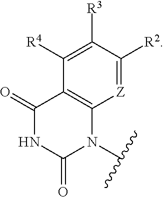

In some embodiments, the compounds described herein are represented by Formula (I), or a pharmaceutically acceptable salt thereof as described in all the embodiments above, wherein one or both of B and B′ is phenyl, which is unsubstituted or substituted with from 1 to 3 R⁷.

In some embodiments, the compounds described herein are represented by Formula (I), or a pharmaceutically acceptable salt thereof as described in all the embodiments above, wherein both of B and B′ is phenyl, which is unsubstituted or substituted with from 1 to 3 R⁷.

In some embodiments, the compounds described herein are represented by Formula (I), or a pharmaceutically acceptable salt thereof as described in all the embodiments above, wherein one or both of B and B′ is a 5- or 6-membered heteroaryl group having from one to three heteroatoms as ring vertices selected from N, O and S; each of which is unsubstituted or substituted with from 1 to 3 R⁷.

In some embodiments, the compounds described herein are represented by Formula (I), or a pharmaceutically acceptable salt thereof as described in all the embodiments above, wherein one or both of B and B′ is selected from the group consisting of thiazolyl, imidazolyl, pyrazolyl, and thienyl; each of which is unsubstituted or substituted with from 1 to 3 R⁷.

In some embodiments, the compounds described herein are represented by Formula (I), or a pharmaceutically acceptable salt thereof as described in all the embodiments above, wherein B is phenyl, and B′ is a 5- or 6-membered heteroaryl group having from one to three heteroatoms as ring vertices selected from N, O and S; each of which is unsubstituted or substituted with from 1 to 3 R⁷.

In some embodiments, the compounds described herein are represented by Formula (I), or a pharmaceutically acceptable salt thereof as described in all the embodiments above, wherein B is phenyl, and B′ is selected from the group consisting of thiazolyl, imidazolyl, pyrazolyl, and thienyl; each of which is unsubstituted or substituted with from 1 to 3 R⁷.

In some embodiments, the compounds described herein are represented by Formula (I), or a pharmaceutically acceptable salt thereof as described in all the embodiments above, wherein X and Y are not both NH. In some embodiments, the compounds described herein are represented by Formula (I), or a pharmaceutically acceptable salt thereof as described in all the embodiments above, wherein X and Y are not both O. In some embodiments, the compounds described herein are represented by Formula (I), or a pharmaceutically acceptable salt thereof as described in all the embodiments above, wherein X and Y are not both S,

In some embodiments, the compounds described herein are represented by Formula (I), or a pharmaceutically acceptable salt thereof as described in all the embodiments above, wherein X is not N(R⁸) and Y is not N(R⁹).

In some embodiments, the compounds described herein are represented by Formula (I), or a pharmaceutically acceptable salt thereof as described in all the embodiments above, wherein X is selected from the group consisting of CH₂, C(O), NH, and O; Y is selected from the group consisting of CH₂, C(O), NH, and O; and wherein X and Y are not both C(O), and at least one of X and Y is CH₂ or C(O).

In some embodiments, the compounds described herein are represented by Formula (I), or a pharmaceutically acceptable salt thereof as described in all the embodiments above, wherein X and Y are combined to form —C(O)NH—.

In some embodiments, the compounds described herein are represented by Formula (I), or a pharmaceutically acceptable salt thereof as described in all the embodiments above, wherein X and Y are combined to form —NHC(O)—.

In some embodiments, the compounds described herein are represented by Formula (I), or a pharmaceutically acceptable salt thereof as described in all the embodiments above, wherein X and Y are combined to form —CH₂NH—.

In some embodiments, the compounds described herein are represented by Formula (I), or a pharmaceutically acceptable salt thereof as described in all the embodiments above, wherein X and Y are combined to form —NHCH₂—.

In some embodiments, the compounds described herein are represented by Formula (I), or a pharmaceutically acceptable salt thereof as described in all the embodiments above, wherein X and Y are combined to form —CH₂O—.

In some embodiments, the compounds described herein are represented by Formula (I), or a pharmaceutically acceptable salt thereof as described in all the embodiments above, wherein X and Y are combined to form —OCH₂—.

In some embodiments of Formula (I) and relevant subembodmients thereof, each R¹, R², R³ and R⁴ are each independently selected from the group consisting of H, C₁₋₆ alkyl, C₁₋₆ haloalkyl, and halo. In some embodiments of Formula (I) and relevant subembodmients thereof, only one of R¹, R², R³ and R⁴ is other than H. In some embodiments of Formula (I) and relevant subembodmients thereof, each of R⁵ and R⁶ is methyl. In some embodiments of Formula (I) and relevant subembodmients thereof, each of R⁵ and R⁶ is hydrogen.

In some embodiments of Formula (I) and relevant subembodmients thereof, each R⁷, when present, is independently halogen or C₁₋₃ alkyl. In some embodiments of Formula (I) and relevant subembodmients thereof, each R⁷, when present, is independently fluoro or methyl.

Representative compounds of Formula (I) are provided in the Examples below.

### General Synthesis

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) and the subembodiments described herein, can be prepared the methods provided in the Examples and adapted similar procedures.

### Utility

As described above, compounds of Formula (I) are useful for treating a disease mediated by MAT2A in a patient comprising administering to the patient a therapeutically effective amount of the compound.

In a related aspect, provided herein are methods of treating a MTAP null cancer in a patient comprising administering to the patient a therapeutically effective amount of a compound of Formula (I).

In another related aspect, provided herein are methods for treating a cancer in a patient, wherein the cancer is characterized by a reduction or absence of MTAP gene expression, the absence of the MTAP gene, reduced function of MTAP protein, reduced level or absence of MTAP protein, MTA accumulation, or combination thereof, comprising administering to the subject a therapeutically effective amount of a compound of Formula (I).

Overexpression of the enzyme MAT2A has been demonstrated to mediate certain cancers. In an embodiment, the cancer is neuroblastoma, intestine carcinoma (such as rectum carcinoma, colon carcinoma, familiary adenomatous polyposis carcinoma and hereditary non-polyposis colorectal cancer), esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors (such as glioblastoma, astrocytoma, meningioma, medulloblastoma and peripheral neuroectodermal tumors), Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

In another embodiment, the cancer is lung cancer, non-small cell lung (NSLC) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the vagina, carcinoma of the vulva, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. In still another embodiment, the cancer is a solid malignant tumor. In another embodiment, the cancer is a tumor having an MTAP gene deletion. In any one of the embodiments herein, the cancer is a solid tumor or a hematological cancer. In one embodiment, the tumor is deficient in MTAP. In another embodiment, the tumor is normal in its expression of MTAP.

Methylthioadenosine phosphorylase (MTAP) is an enzyme found in all normal tissues that catalyzes the conversion of methylthioadenosine (MTA) into adenine and 5-methylthio-ribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

Many human and murine malignant cells lack MTAP activity. MTAP deficiency is not only found in tissue culture cells but the deficiency is also present in primary leukemias, gliomas, melanomas, pancreatic cancers, non-small cell lung cancers (NSLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas. It has been reported by K. Marjon et al., Cell Reports 15 (2016) 574-587, incorporated herein by reference, that proliferation of cancer cells that are MTAP null is inhibited by knocking down MAT2A expression with shRNA. An MTAP null cancer is a cancer in which the MTAP gene has been deleted or lost or otherwise deactivated or a cancer in which the MTAP protein has a reduced or impaired function.

Accordingly, in an embodiment of the present disclosure there is provided a method for treating an MTAP null cancer in a patient wherein said cancer is characterized by a reduction or absence of MTAP expression or absence of the MTAP gene or reduced function of MTAP protein, reduced level or absence of MTAP protein, MTA accumulation, or combination thereof as compared to cancers where the MTAP gene is present and fully functioning, said method comprising administering to the patient in need thereof a therapeutically effective amount of a compound of Formula (I) or a subembodiment described herein or a pharmaceutically acceptable salt thereof. In another embodiment, provided is a method of treating an MTAP null cancer in a patient comprising administering to the patient in need thereof an effective amount of a compound of Formula (I) or a subembodiment described herein or a pharmaceutically acceptable salt thereof. In an embodiment, the MTAP null cancer is leukemia, glioma, melanoma, pancreatic, non-small cell lung cancer (NSLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma or mesothelioma. In another embodiment, the MTAP null cancer is pancreatic cancer. In yet another embodiment, the MTAP null cancer is bladder cancer, melanoma, brain cancer, lung cancer, pancreatic cancer, breast cancer, esophageal cancer, head and neck cancer, kidney cancer, colon cancer, diffuse large B cell lymphoma (DLBCL), acute lymphoblastic leukemia (ALL) or mantle cell lymphoma (MCL). In yet another embodiment, the MTAP null cancer is gastric cancer. In yet another embodiment, the cancer is colon cancer. In yet another embodiment, the MTAP null cancer is liver cancer. In yet another embodiment, the MTAP null cancer is glioblastoma multiforme (GBM). In yet another embodiment, the MTAP null cancer is bladder cancer. In yet another embodiment, the MTAP null cancer is esophageal cancer. In yet another embodiment, the MTAP null cancer is breast cancer. In yet another embodiment, the MTAP null cancer is NSLCC. In yet another embodiment, the MTAP null cancer is MCL. In yet another embodiment, the MTAP null cancer is DLBCL. In yet another embodiment, the MTAP null cancer is ALL.

Genomic analysis of MTAP null cell lines has shown that cell lines that also incorporate a KRAS mutation or a p53 mutation were sensitive to MAT2A inhibition.

Accordingly, also provided is a method for treating a cancer in a patient wherein said cancer is characterized by reduction or absence of MTAP expression or absence of the MTAP gene or reduced function of MTAP protein (i.e, MTAP null) and further characterized by the presence of mutant KRAS and/or mutant p53, said method comprising administering to the patient a therapeutically effective amount of a compound of Formula (I) or a subembodiment described herein. In one embodiment, the cancer is MTAP null and KRAS mutant. In another embodiment, the cancer is MTAP null and p53 mutant. In yet another embodiment, the cancer is MTAP null, KRAS mutant and p53 mutant.

The term “mutant KRAS” or “KRAS mutation” refers to KRAS protein (or gene encoding said protein) incorporating an activating mutation that alters its normal function. For example, a mutant KRAS protein may incorporate a single amino acid substitution at position 12 or 13. In a particular embodiment, the KRAS mutant incorporates a G12×or G13X substitution, wherein X represents any amino acid change at the indicated position. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. In an embodiment, said p53 mutation is, Y126_splice, K132Q, M133K, R174fs, R175H, R196*, C238S, C242Y, G245S, R248W, R248Q, 1255T, D259V, S261_splice, R267P, R273C, R282W, A159V or R280K. In an embodiment, the foregoing cancer is non-small cell lung cancer (NSLCC), pancreatic cancer, head and neck cancer, gastric cancer, breast cancer, colon cancer or ovarian cancer.

### Assay

The ability of compounds of the disclosure to inhibit MAT2A can be measured as described in Biological Example 1 below.

### Pharmaceutical Composition

The compounds of Formula (I) or a subembodiment described herein, or a pharmaceutically acceptable salt thereof, may be in the form of compositions suitable for administration to a subject. In general, such compositions are pharmaceutical compositions comprising a compound of Formula (I) or a subembodiment described herein or a pharmaceutically acceptable salt thereof and one or more pharmaceutically acceptable or physiologically acceptable excipients. In certain embodiments, the compound of Formula (I) or a subembodiment described herein, or a pharmaceutically acceptable salt thereof is present in a therapeutically effective amount. The pharmaceutical compositions may be used in the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., a compound of Formula (I) or a subembodiment described herein, a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound of Formula (I) or a subembodiment described herein, or a salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a compound of Formula (I) or a subembodiment described herein, or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound of Formula (I) or a subembodiment described herein, or a salt thereof over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

A compound of Formula (I) or a subembodiment described herein, or a salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

### Routes of Administration

Compounds of Formula (I) or a subembodiment described herein, or a salt thereof and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to administer the compounds of Formula (I) or a subembodiment described herein, or a salt thereof over a defined period of time. Particular embodiments of the present invention contemplate oral administration.

### Combination Therapy

The present invention contemplates the use of compounds of Formula (I) or a subembodiment described herein, or a salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the compounds of Formula (I) or a subembodiment described herein, or a salt thereof are administered or applied sequentially, e.g., where one agent is administered prior to one or more other agents. In other embodiments, the compounds of Formula (I) or a subembodiment described herein, or a salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The compounds of Formula (I) or a subembodiment described herein, or a salt thereof may be used in combination with at least one other (active) agent in any manner appropriate under the circumstances. In one embodiment, treatment with the at least one active agent and at least one compound of Formula (I) or a subembodiment described herein, or a salt thereof is maintained over a period of time. In another embodiment, treatment with the at least one active agent is reduced or discontinued (e.g., when the subject is stable), while treatment with the compound of Formula (I) or a subembodiment described herein, or a salt thereof is maintained at a constant dosing regimen. In a further embodiment, treatment with the at least one active agent is reduced or discontinued (e.g., when the subject is stable), while treatment with a compound of Formula (I) or a subembodiment described herein, or a salt thereof is reduced (e.g., lower dose, less frequent dosing or shorter treatment regimen). In yet another embodiment, treatment with the at least one active agent is reduced or discontinued (e.g., when the subject is stable), and treatment with the compound of Formula (I) or a subembodiment described herein, or a salt thereof is increased (e.g., higher dose, more frequent dosing or longer treatment regimen). In yet another embodiment, treatment with the at least one active agent is maintained and treatment with the compound of Formula (I) or a subembodiment described herein, or a salt thereof is reduced or discontinued (e.g., lower dose, less frequent dosing or shorter treatment regimen). In yet another embodiment, treatment with the at least one active agent and treatment with the compound of Formula (I) or a subembodiment described herein, or a salt thereof are reduced or discontinued (e.g., lower dose, less frequent dosing or shorter treatment regimen).

The present disclosure provides methods for treating cancer with a compound of Formula (I) or a subembodiment described herein, or a salt thereof and at least one additional therapeutic or diagnostic agent.

In some embodiments, the compound of Formula (I) or a subembodiment described herein, or a salt thereof is administered in combination with at least one additional therapeutic agent, selected from Temozolomide, Pemetrexed, Pegylated liposomal doxorubicin (Doxil), Eribulin (Halaven), Ixabepilone (Ixempra), Protein-bound paclitaxel (Abraxane), Oxaliplatin, Irinotecan, Venatoclax (bcl2 inhibitor), 5-azacytadine, Anti-CD20 therapeutics, such as Rituxan and obinutuzumab, Hormonal agents (anastrozole, exemestand, letrozole, zoladex, lupon eligard), CDK4/6 inhibitors, Palbociclib, Abemaciclib, CPI (Avelumab, Cemiplimab-rwlc, and Bevacizumab.

In certain embodiments, the present disclosure provides methods for treating cancer comprising administration of a compound of Formula (I) or a subembodiment described herein, or a salt thereof in combination with a signal transduction inhibitor (STI) to achieve additive or synergistic suppression of tumor growth. As used herein, the term “signal transduction inhibitor” refers to an agent that selectively inhibits one or more steps in a signaling pathway. Examples of signal transduction inhibitors (STIs) useful in methods described herein include, but are not limited to: (i) bcr/abl kinase inhibitors (e.g., GLEEVEC); (ii) epidermal growth factor (EGF) receptor inhibitors, including kinase inhibitors and antibodies; (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with one or more compounds of Formula (I) or a subembodiment described herein, or a salt thereof for the suppression of tumor growth in cancer patients.

In certain embodiments, the present disclosure provides methods for treating cancer comprising administration of a compound of Formula (I) or a subembodiment described herein, or a salt thereof in combination with a chemotherapeutic agents. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above. In a particular embodiment, compounds of the present disclosure are coadministered with a cytostatic compound selected from the group consisting of cisplatin, doxorubicin, taxol, taxotere and mitomycin C. In a particular embodiment, the cytostatic compound is doxorubicin.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, enzalutamide, apalutamide, abiraterone acetate, leuprolide, and goserelin; and pharmaceutically acceptable salts, acids or derivatives of any of the above. In certain embodiments, combination therapy comprises administration of a hormone or related hormonal agent.

The present disclosure also contemplates the use of the compounds of Formula (I) or a subembodiment described herein, or a salt thereof in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGFβ, CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure contemplates the use of compounds of Formula (I) or a subembodiment described herein, or a salt thereof in combination with inhibitors of the aforementioned immune-checkpoint receptors and ligands, for example ipilimumab, abatacept, nivolumab, pembrolizumab, atezolizumab, nivolumab, and durvalumab.

Additional treatment modalities that may be used in combination with a compound of Formula (I) or a subembodiment described herein, or a salt thereof include radiotherapy, a monoclonal antibody against a tumor antigen, a complex of a monoclonal antibody and toxin, a T-cell adjuvant, bone marrow transplant, or antigen presenting cells (e.g., dendritic cell therapy).

The present disclosure contemplates the use of compounds of Formula (I) or a subembodiment described herein, or a salt thereof for the treatment of glioblastoma either alone or in combination with radiation and/or temozolomide (TMZ), avastin or lomustine.

The present disclosure encompasses pharmaceutically acceptable salts, acids or derivatives of any of the above.

### Dosing

The compounds of Formula (I) or a subembodiment described herein, or a salt thereof may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or EDso of an agent is the dose or amount of an agent that produces a therapeutic response or desired effect in 50% of the population to which it is administered. Although the EDso is commonly used as a measure of reasonable expectance of an agent's effect, it is not necessarily the dose that a clinician might deem appropriate taking into consideration all relevant factors. Thus, in some situations the effective amount is more than the calculated EDso, in other situations the effective amount is less than the calculated EDso, and in still other situations the effective amount is the same as the calculated EDso.

In addition, an effective dose of a compound of Formula (I) or a subembodiment described herein, or a salt thereof may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the compounds of Formula (I) or a subembodiment described herein, or a salt thereof may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the compound of Formula (I) or a subembodiment described herein, or a salt thereof is contained in a “unit dosage form”. The phrase “unit dosage form” refers to physically discrete units, each unit containing a predetermined amount of the compound of Formula (I) or a subembodiment described herein, or a salt thereof, either alone or in combination with one or more additional agents, sufficient to produce the desired effect. It will be appreciated that the parameters of a unit dosage form will depend on the particular agent and the effect to be achieved.

### Kits

The present invention also contemplates kits comprising a compound of Formula (I) or a subembodiment described herein, or a salt thereof, and pharmaceutical compositions thereof. The kits are generally in the form of a physical structure housing various components, as described below, and may be utilized, for example, in practicing the methods described above.

A kit can include one or more of the compound of Formula (I) or a subembodiment described herein, or a salt thereof (provided in, e.g., a sterile container), which may be in the form of a pharmaceutical composition suitable for administration to a subject. The compound of Formula (I) or a subembodiment described herein, or a salt thereof can be provided in a form that is ready for use (e.g., a tablet or capsule) or in a form requiring, for example, reconstitution or dilution (e.g., a powder) prior to administration. When the compounds of Formula (I) or a subembodiment described herein, or a salt thereof are in a form that needs to be reconstituted or diluted by a user, the kit may also include diluents (e.g., sterile water), buffers, pharmaceutically acceptable excipients, and the like, packaged with or separately from the compounds of Formula (I) or a subembodiment described herein, for a salt thereof. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EMBODIMENTS

Embodiment 1. A compound having the Formula (I)

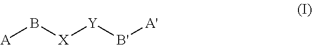

or a pharmaceutically acceptable salt thereof, wherein
A and A′ are each independently selected from the group consisting of:

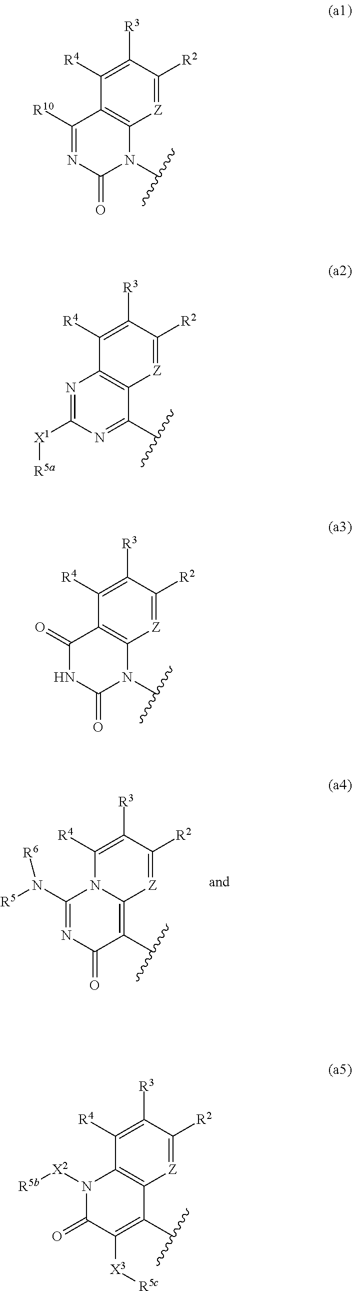

wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{Y}, —C(O)R^{y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

Embodiment 2. The compound of embodiment 1, or a pharmaceutically acceptable salt thereof, wherein
A and A′ are each independently selected from the group consisting of:

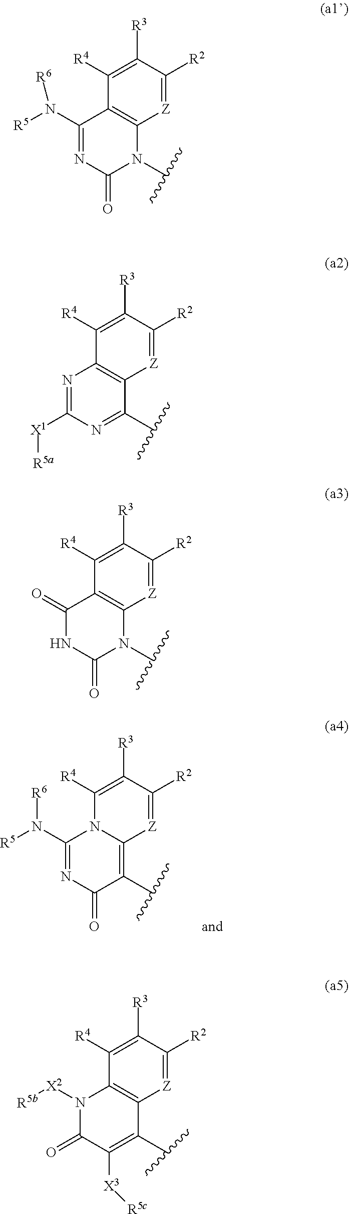

wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(CH₃), O and S;
Y is selected from the group consisting of CH₂, C(O), NH, N(CH₃), O and S;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R⁵ and R⁶ are each independently selected from the group consisting of H, C₁₋₆ alkyl, C₁₋₆ haloalkyl, and C₃₋₆ cycloalkyl;
each R⁷ is independently selected from the group consisting of halogen, cyano, C₁₋₃ alkyl, and C₁₋₃ haloalkyl;
X¹ is selected from the group consisting of a bond, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond and C₁₋₄ alkylene;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁-6 alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{Y}, —C(O)R^{y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

Embodiment 3. The compound of embodiment 1 or 2, or a pharmaceutically acceptable salt thereof, wherein each A and A′ has the formula:

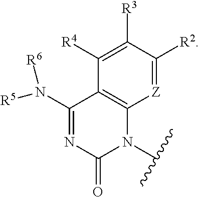

Embodiment 4. The compound of embodiment 1 or 2, or a pharmaceutically acceptable salt thereof, wherein each A and A′ has the formula:

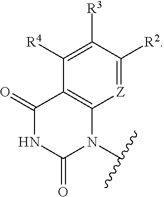

Embodiment 5. The compound of embodiment 1 or 2, or a pharmaceutically acceptable salt thereof, wherein only one of A and A′ has the formula:

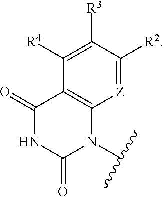

Embodiment 6. The compound of any one of embodiments 1 to 5, or a pharmaceutically acceptable salt thereof, wherein one or both of B and B′ is phenyl, which is unsubstituted or substituted with from 1 to 3 R⁷.

Embodiment 7. The compound of any one of embodiments 1 to 5, or a pharmaceutically acceptable salt thereof, wherein both of B and B′ are phenyl, which is unsubstituted or substituted with from 1 to 3 R⁷.

Embodiment 8. The compound of any one of embodiments 1 to 5, or a pharmaceutically acceptable salt thereof, wherein one or both of B and B′ is a 5- or 6-membered heteroaryl group having from one to three heteroatoms as ring vertices selected from N, O and S; and is unsubstituted or substituted with from 1 to 3 R⁷.

Embodiment 9. The compound of any one of embodiments 1 to 5, or a pharmaceutically acceptable salt thereof, wherein one or both of B and B′ is selected from the group consisting of thiazolyl, imidazolyl, pyrazolyl, and thienyl; and is unsubstituted or substituted with from 1 to 3 R⁷.

Embodiment 10. The compound of any one of embodiments 1 to 5, or a pharmaceutically acceptable salt thereof, wherein B is phenyl, and B′ is 5- or 6-membered heteroaryl group having from one to three heteroatoms as ring vertices selected from N, O and S; and is unsubstituted or substituted with from 1 to 3 R⁷.

Embodiment 11. The compound of any one of embodiments 1 to 5, or a pharmaceutically acceptable salt thereof, wherein B is phenyl, and B′ is thiazolyl, imidazolyl, pyrazolyl, and thienyl; and is unsubstituted or substituted with from 1 to 3 R⁷.

Embodiment 12. The compound of embodiment 1 to 11, or a pharmaceutically acceptable salt thereof, wherein X and Y are combined to form —C(O)NH—.

Embodiment 13. The compound of embodiment 1 to 11, or a pharmaceutically acceptable salt thereof, wherein X and Y are combined to form —NHC(O)—.

Embodiment 14. The compound of embodiment 1 to 11, or a pharmaceutically acceptable salt thereof, wherein X and Y are combined to form —CH₂NH—.

Embodiment 15. The compound of embodiment 1 to 11, or a pharmaceutically acceptable salt thereof, wherein X and Y are combined to form —NHCH₂—.

Embodiment 16. The compound of embodiment 1 to 11, or a pharmaceutically acceptable salt thereof, wherein X and Y are combined to form —CH₂O—.

Embodiment 17. The compound of embodiment 1 to 11, or a pharmaceutically acceptable salt thereof, wherein X and Y are combined to form —OCH₂—.

Embodiment 18. The compound of any one of embodiments 1 to 17, or a pharmaceutically acceptable salt thereof, wherein each R¹, R², R³ and R⁴ are each independently selected from the group consisting of H, C₁₋₆ alkyl, C₁₋₆ haloalkyl, and halo.

Embodiment 19. The compound of embodiment 18, or a pharmaceutically acceptable salt thereof, wherein only one of R¹, R², R³ and R⁴ is other than H.

Embodiment 20. The compound of any one of embodiments 1 to 19, or a pharmaceutically acceptable salt thereof, wherein each of R⁵ and R⁶ is methyl.

Embodiment 21. The compound of any one of embodiments 1 to 19, or a pharmaceutically acceptable salt thereof, wherein one of B and B′ is unsubstituted.

Embodiment 22. The compound of any one of embodiments 1 to 19, or a pharmaceutically acceptable salt thereof, wherein both B and B′ are unsubstituted.

Embodiment 23. The compound of any one of embodiments 1 to 19 and 20A, or a pharmaceutically acceptable salt thereof, wherein one of B and B′ is unsubstituted and the other is substituted with 1 R⁷, wherein R⁷ is halogen or C₁₋₃ alkyl.

Embodiment 24. The compound of any one of embodiments 1 to 19, or a pharmaceutically acceptable salt thereof, wherein both of B and B′ are each independently substituted with 1 R⁷, wherein R⁷ is halogen or C₁₋₃ alkyl.

Embodiment 25. A pharmaceutical composition comprising a compound of any one of embodiments 1 to 24, or a pharmaceutically acceptable salt thereof and at least one pharmaceutically acceptable excipient

Embodiment 26. A method for treating a disease mediated by MAT2A in a patient comprising administering to the patient a therapeutically effective amount of a compound of any one of embodiments 1 to 24 or a pharmaceutical composition of embodiment 25.

Embodiment 27. The method of embodiment 16, wherein the disease is cancer.

Embodiment 28. A method of treating a MTAP null cancer in a patient comprising administering to the patient a therapeutically effective amount of a compound of any one of embodiments 1 to 24 or a pharmaceutical composition of embodiment 25.

Embodiment 29. A method for treating a cancer in a patient, wherein the cancer is characterized by a reduction or absence of MTAP gene expression, the absence of the MTAP gene, reduced function of MTAP protein, reduced level or absence of MTAP protein, MTA accumulation, or combination thereof, comprising administering to the subject a therapeutically effective amount of a compound of any one of embodiments 1 to 24 or a pharmaceutical composition of embodiment 25.

Embodiment 30. The method of any one of embodiments 26 to 29, wherein the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophagogastric cancer, malignant peripheral nerve sheath tumor, and mesothelioma.

Embodiment 31. Use of a compound or a pharmaceutically acceptable salt thereof of any one of embodiments 1 to 24 or a pharmaceutical composition of embodiment 25 in the manufacture of a medicament for use in the treatment of cancer.

Embodiment 32. The method or use of any one of embodiments 26 to 29 and 31, wherein the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophageal cancer, malignant peripheral nerve sheath tumor, leiomyosarcoma, cholangiocarcinoma, and mesothelioma.

### EXAMPLES

The following examples and references (intermediates) are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: μg=microgram; μl or μL=microliter; mM=millimolar; μM=micromolar; aa=amino acid(s); Ac₂O=acetic anhydride; AcCl=acetylchloride; ACN=acetonitrile; AIBN=2,2′-Azobis(2-methylpropionitrile); BID=twice daily; BINAP=2,2′-bis(diphenylphosphino)-1 ,1 ′-binaphthyl; Boc₂O or (Boc)₂O=di-tert-butyl dicarbonate; bp=base pair(s); BSA=bovine serum albumin; BW=body weight; d=doublet; dd=doublet of doublets; DEAD=diethyl azodicarboxylate; DIBAL=diisobutylaluminium hydride DIEA=N,N-diisopropylethylamine; DIPEA=N,N-diisopropylethylamine; dl or dL=deciliter; DMA=dimethylacetamide; DMAP=dimethylaminopyridine; DME=1,2-dimethoxyethane; DMEM=Dulbeco's Modification of Eagle's Medium; DMF=N,N-dimethylformamide; DMSO=dimethylsulfoxide; dppf=1,1′-Bis(diphenylphosphino)ferrocene; DTT=dithiothreitol; EDTA=ethylenediaminetetraacetic acid; ES=electrospray; EtOAc=ethyl acetate; EtOH=ethanol; g=gram; h or hr=hour(s); HATU=2-(1 H-7-azabenzotriazol-1-yl)-1,1,3,3-tetramethyluronium hexafluorophosphate; HEPES=4-(2-hydroxyethyl)-1-piperazineethylanesulfonic acid; HOAc=acetic acid; HPLC=high performance liquid chromatography; HPLC=high pressure liquid chromatography; i.m.=intramuscular(ly); i.p.=intraperitoneal(ly); IHC=immunohistochemistry; IPA=isopropyl alcohol; kb=kilobase(s); kDa=kilodalton; kg=kilogram; 1 or L=liter; LC=liquid chromatography; LCMS=liquid chromatography and mass spectrometry; m/z=mass to charge ratio; M=molar; m=multiplet; MeCN=acetonitrile; MeOH=methanol; MeSO₂Cl=methanesulfonylchloride; mg=milligram; min=minute(s); min=minutes; ml or mL=milliliter; mM=millimolar; MS=mass spectrometry; MsC1=methanesulfonylchloride; N=normal; NADPH=nicotinamide adenine dinucleotide phosphate; NBS═N-bromosuccinamide; ng=nanogram; nm=nanometer; nM=nanomolar; NMP=N-methylpyrrolidone; NMR=nuclear magnetic resonance; ns=not statistically significant; nt=nucleotides(s); PBS=phosphate-buffered saline; Pd/C=palladium on carbon; Pd₂(dba)₃=Tris(debenzylideneactone) dipalladium; Pd(dppf)C12=1,1′-bis(diphenylphosphino)ferrocene-palladium(ll)dichloride; PE=petroleum ether; QD=daily; QM=monthly; QW=weekly; rac=racemic; Rt=retention time; s=singlet; s or see=second(s); sat.=saturated; SC or SQ=subcutaneous(ly); t=triplet; TBAB=tetra-n-butylammonium bromide; TEA=triethylamine; TFA=trifluoroacetic acid; THF=tetrahydrofuran; TLC=thin layer chromatography; TMSC1=trimethylsilylchloride; TsOH=p-toluenesulfonic acid; U=unit; wt=wildtype.

### SYNTHETIC EXAMPLES

### Example 1

### 5-(7-Chloro-4-(dimethylamino)-2-oxoquinazolin-1(2H)-yl)-N-(3-(7-chloro-4-(dimethylamino)-2-oxoquinazolin-1(2H)-yl)phenyl)thiophene-3-carboxamide

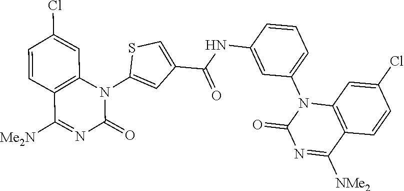 [SMILES: CN(C)c1nc(=O)n(-c2cccc(NC(=O)c3csc(-n4c(=O)nc(N(C)C)c5ccc(Cl)cc54)c3)c2)c2cc(Cl)ccc12]

### Step 1

### tert-Butyl (3-(3-(4-chloro-2-fluorobenzoyl)ureido)phenyl)carbamate

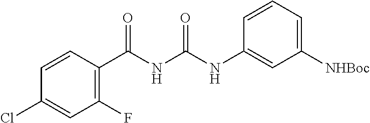 [SMILES: CC(C)(C)OC(=O)Nc1cccc(NC(=O)NC(=O)c2ccc(Cl)cc2F)c1]

To a stirred solution of 4-chloro-2-fluorobenzamide (1.65 g, 9.56 mmol, 1.00 equiv) in DCE (10 mL) was added oxalyl chloride (1.58 g, 12.43 mmol, 1.30 equiv) at RT and the reaction mixture was stirred at 55° C. for 1 h and at 85° C. for 4 h. The reaction mixture was concentrated under reduced pressure to afford the corresponding isocyanate. The isocyanate was dissolved in DCE (10 mL) and added to a solution of tert-butyl (3-aminophenyl)carbamate (2.0 g, 9.56 mmol, 1.00 equiv) in DCE (10 mL) at 0° C. The reaction mixture was stirred at RT for 2 h. The reaction mixture was diluted with Et₂O (50 mL), filtered and dried under vacuum to afford tert-butyl (3-(3-(4-chloro-2-fluorobenzoyl)ureido)phenyl)carbamate (3.0 g, 81%) as an off-white solid. LC-MS (ESI, m/z): 408.2 [M+H]⁺.

### Step 2

### tert-Butyl (3-(7-chloro-2,4-dioxo-3,4-dihydroquinazolin-1(2H)-yl)phenyl)carbamate

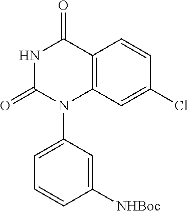 [SMILES: CC(C)(C)OC(=O)Nc1cccc(-n2c(=O)[nH]c(=O)c3ccc(Cl)cc32)c1]

To a stirred solution of tert-butyl (3-(3-(4-chloro-2-fluorobenzoyl)ureido)phenyl)carbamate (2.9 g, 7.12 mmol, 1.00 equiv) in THF (150 mL) was added KO^{t}Bu (2.40 g, 21.4 mmol, 3.00 equiv) at 0° C. and the reaction was stirred at 100° C. for 1 h. The reaction mixture was diluted with water (100 mL) and extracted with EtOAc (2×100 mL). The combined organic layers were washed with brine (100 mL), dried over Na₂SO₄, filtered and concentrated to afford tert-butyl (3-(7-chloro-2,4-dioxo-3,4-dihydroquinazolin-1(2H)-yl)phenyl)carbamate (2.2 g, 79%) as a light brown solid. LC-MS (ESI, m/z): 332.1 [(M+H)−56]⁺.

### Step 3

### tert-Butyl (3-(7-chloro-4-(dimethylamino)-2-oxoquinazolin-1(2H)-yl)phenyl)carbamate

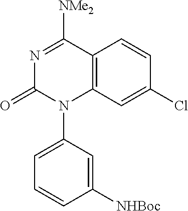 [SMILES: CN(C)c1nc(=O)n(-c2cccc(NC(=O)OC(C)(C)C)c2)c2cc(Cl)ccc12]

To a stirred solution of tert-butyl (3-(7-chloro-2,4-dioxo-3,4-dihydroquinazolin-1(2H)-yl)phenyl)carbamate (1.0 g, 2.58 mmol, 1.00 equiv) in MeCN (10 mL) was added Et₃N (1.08 mL, 7.74 mmol, 3.00 equiv), DMAP (0.63 g, 5.16 mmol, 2.00 equiv) and 2,4,6-triisopropylbenzenesulfonyl chloride (1.56 g, 5.16 mmol, 2.00 equiv) at 0° C. and the reaction mixture was stirred at 80° C. for 4 h. The reaction mixture was cooled to 0° C. and Me₂NH (12.9 mL, 12.9 mmol, 5.00 equiv, 1.0 M in THF) was added and the mixture was stirred at RT for 2 h. The reaction mixture was diluted with water (30 mL) and extracted with EtOAc (2×30 mL). The combined organic layers were washed with brine (20 mL), dried over anhydrous Na₂SO₄, filtered and concentrated. Purification by silica gel chromatography afforded tert-butyl (3-(7-chloro-4-(dimethylamino)-2-oxoquinazolin-1(2H)-yl)phenyl)carbamate (0.35 g, 35%) as a brown solid. LC-MS (ESI, m/z): 415.2 [M+H]⁺.

### Step 4

### 1-(3-Aminophenyl)-7-chloro-4-(dimethylamino)quinazolin-2(1H)-one

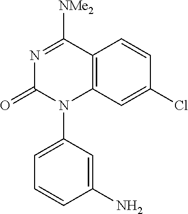 [SMILES: CN(C)c1nc(=O)n(-c2cccc(N)c2)c2cc(Cl)ccc12]

To a solution of tert-butyl (3-(7-chloro-4-(dimethylamino)-2-oxoquinazolin-1(2H)-yl)phenyl)carbamate (0.7 g, 1.7 mmol, 1.0 equiv) in DCM (5 mL) was added TFA (0.26 mL, 3.4 mmol, 2.0 equiv) at 0° C. and the mixture was stirred for RT for 16 h. All the volatiles were removed under reduced pressure, the residue was diluted with water (50 mL) and extracted with EtOAc (2×30 mL). The combined organic layers were washed with saturated NaHCO₃ (20 mL), dried over anhydrous Na₂SO₄, filtered and concentrated to afford 1-(1-(1H-imidazol-4-yl)ethyl)-7-bromo-4-(dimethylamino)quinazolin-2(1H)-one (0.4 g, 75%) as a light yellow solid. LC-MS (ESI, m/z): 315.1 [M+H]⁺.

### Step 5

### Methyl 5-nitrothiophene-3-carboxylate

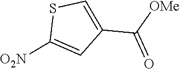 [SMILES: COC(=O)c1csc([N+](=O)[O-])c1]

To a stirred solution of 5-nitrothiophene-3-carboxylic acid (10 g, 57.8 mmol, 1.00 equiv) in MeOH (100 mL) was added H₂SO₄ (2.0 mL) at RT and the reaction mixture was stirred for 16 h at RT. The reaction mixture was diluted with water (100 mL) and extracted with EtOAc (2×200 mL). The combined organic layers were washed with brine (100 mL), dried over Na₂SO₄, filtered and concentrated to afford methyl 5-nitrothiophene-3-carboxylate (9.0 g, 84%) as an off-white solid. LC-MS (ESI, m/z): 188.0 [M+H]⁺.

### Step 6

### Methyl 5-aminothiophene-3-carboxylate

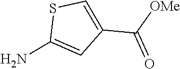

To a stirred solution of methyl 5-nitrothiophene-3-carboxylate (8.0 g, 46.2 mmol, 1.00 equiv) in EtOAc (80 mL) was added 10% Pd/C (1.6 g, 20% w/w) at RT and the reaction mixture was stirred under hydrogen atmosphere (balloon) for 16 h at RT. The reaction mixture was filtered through a pad of Celite by washing with EtOAc (200 mL) and the filtrate was evaporated to afford methyl 5-aminothiophene-3-carboxylate (6.0 g, 83%) as an off-white solid. LC-MS (ESI, m/z): 158.0 [M+H]⁺.

### Step 7

### Methyl 5-(3-(4-chloro-2-fluorobenzoyl)ureido)thiophene-3-carbox late

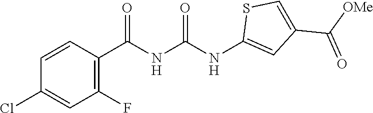 [SMILES: COC(=O)c1csc(NC(=O)NC(=O)c2ccc(Cl)cc2F)c1]

The title compound was prepared by substituting tert-butyl (3-aminophenyl)carbamate with methyl 5-aminothiophene-3-carboxylate in step 1. LC-MS (ESI, m/z): 357.2 [M+H]⁺.

### Step 8

### Methyl 5-(7-chloro-2,4-dioxo-3,4-dihydroquinazolin-1(2H)-yl)thiophene-3-carboxylate

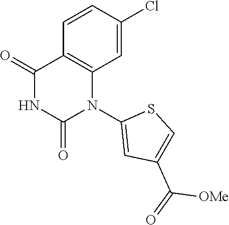 [SMILES: COC(=O)c1csc(-n2c(=O)[nH]c(=O)c3ccc(Cl)cc32)c1]

To a stirred solution of methyl 5-(3-(4-chloro-2-fluorobenzoyl)ureido)thiophene-3-carboxylate (4.0 g, 11.2 mmol, 1.00 equiv) in DMF (200 mL) was added KHMDS (16.9 mL, 16.9 mmol, 1.5 equiv, 1 M in THF) dropwise at 0° C. and the reaction mixture was stirred at 100° C. for 2 h. The reaction mixture was diluted with ice-cold water (100 mL) and acidified with aqueous HCl (25 mL, 1 M). The precipitated solid was filtered by washing with ice-cold water (50 mL) and dried under vacuum to afford methyl 5-(7-chloro-4-hydroxy-2-oxoquinazolin-1(2H)-yl)thiophene-3-carboxylate (2.0 g, 52%) as an off-white solid. LC-MS (ESI, m/z): 336.9 [M+H]⁺.

### Step 9

### Methyl 5-(7-chloro-4-(dimethylamino)-2-oxoquinazolin-1(2H)-yl)thiophene-3-carboxylate

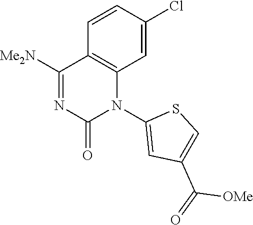 [SMILES: COC(=O)c1csc(-n2c(=O)nc(N(C)C)c3ccc(Cl)cc32)c1]

To a stirred solution of methyl 5-(7-chloro-4-hydroxy-2-oxoquinazolin-1(2H)-yl)thiophene-3-carboxylate (3.0 g, 8.9 mmol, 1.00 equiv) in MeCN (40 mL) were added DIPEA (6.20 mL, 35.6 mmol, 4.00 equiv) and POCl₃ (2.73 g, 17.8 mmol, 2.00 equiv) at 0° C. and the reaction mixture was stirred at 100° C. for 4 h. The reaction mixture was poured into ice-cold water (60 mL) and extracted with Et₂O (2×40 mL). The combined organic layers were washed with aqueous saturated NaHCO₃ (30 mL), dried over Na₂SO₄, filtered and concentrated (below 10° C.) to afford methyl 5-(4,7-dichloro-2-oxoquinazolin-1(2H)-yl)thiophene-3-carboxylate (3.0 g).

To a stirred solution of methyl 5-(4,7-dichloro-2-oxoquinazolin-1(2H)-yl)thiophene-3-carboxylate (3.0 g, 8.5 mmol, 1.00 equiv) in MeCN (30 mL) were added TEA (3.03 g, 34 mmol, 4.00 equiv) and Me₂NH (8.5 mL, 17 mmol, 2.0 equiv, 2 M in THF) and the resulting mixture was stirred at 80° C. for 2 h. The reaction mixture was diluted with ice-cold water (50 mL) and extracted with EtOAc (3×50 mL). The combined organic layers were washed with brine (20 mL), dried over anhydrous Na₂SO₄, filtered and concentrated to afford methyl 5-(7-chloro-4-(dimethylamino)-2-oxoquinazolin-1(2H)-yl)thiophene-3-carboxylate (1.5 g, 51%) as a brown solid. LC-MS (ESI, m/z): 364.2 [M+H]⁺.

### Step 10

### 5-(7-Chloro-4-(dimethylamino)-2-oxoquinazolin-1(2H)-yl)thiophene-3-carboxylic acid

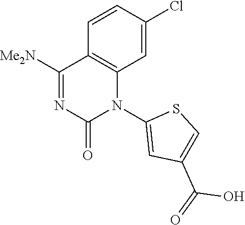 [SMILES: CN(C)c1nc(=O)n(-c2cc(C(=O)O)cs2)c2cc(Cl)ccc12]

To a stirred solution of methyl 5-(7-chloro-4-(dimethylamino)-2-oxoquinazolin-1(2H)-yl)thiophene-3-carboxylate (0.30 g, 0.80 mmol, 1.00 equiv) in DCE (10 mL) was added Me₃SnOH (0.72 g, 4.0 mmol, 5.00 equiv) at RT and the reaction mixture was stirred at 80° C. for 24 h. The reaction mixture was diluted with water (30 mL) and extracted with DCM (2×30 mL). The combined organic layers were washed with brine (30 mL), dried over Na₂SO₄, filtered and concentrated to afford 5-(7-chloro-4-(dimethylamino)-2-oxoquinazolin-1(2H)-yl)thiophene-3-carboxylic acid (0.18 g, 62%) as a light brown solid. LC-MS (ESI, m/z): 350.2 [M+H]⁺.

### Step 11

### 5-(7-Chloro-4-(dimethylamino)-2-oxoquinazolin-1(2H)-yl)-N-(3-(7-chloro-4-(dimethylamino)-2-oxoquinazolin-1(2H)-yl)phenyl)thiophene-3-carboxamide

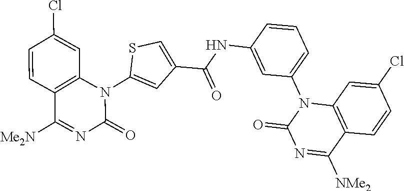 [SMILES: CN(C)c1nc(=O)n(-c2cccc(NC(=O)c3csc(-n4c(=O)nc(N(C)C)c5ccc(Cl)cc54)c3)c2)c2cc(Cl)ccc12]

To a stirred solution of 5-(7-chloro-4-(dimethylamino)-2-oxoquinazolin-1(2H)-yl)thiophene-3-carboxylic acid (0.05 g, 0.1 mmol, 1.00 equiv) in DMF (5 mL) at 0° C. were added DIPEA (0.1 mL, 0.4 mmol, 4.00 equiv), EDC.HCl (0.028 g, 0.15 mmol, 1.5 equiv) and HOBt (0.023 g, 0.15 mmol, 1.5 equiv) and the reaction mixture was stirred at the same temperature for 30 min. Then 1-(3-aminophenyl)-7-chloro-4-(dimethylamino)quinazolin-2(1H)-one (0.031 g, 0.1 mmol, 1.00 equiv) (product from step 4) was added and stirred at 60° C. for 16 h. The reaction mixture was diluted with water (30 mL) and extracted with EtOAc (2×50 mL). The combined organic layers were washed with brine (20 mL), dried over Na₂SO₄, filtered and concentrated. Purification by prep HPLC (mobile phase A—10 mM aqueous ammonium bicarbonate, mobile phase B—MeCN, column—X-BRIDGE C18 (19×250) mm 5mic, flow—18 ml/min, gradient method) gave 5-(7-chloro-4-(dimethylamino)-2-oxoquinazolin-1(2H)-yl)-N-(3-(7-chloro-4-(dimethylamino)-2-oxoquinazolin-1(2H)-yl)phenyl)thiophene-3-carboxamide (14 mg, 15%) as an off-white solid. ¹HNMR (500 MHz, DMSO-d₆) δ 10.36 (s, 1H), 8.48 (d, J=1.5 Hz, 1H), 8.07-8.04 (m, 2H), 7.89-7.86 (m, 1H), 7.73-7.71 (m, 1H), 7.62-7.56 (m, 2H), 7.27 (dd, J=9.0, 2.0 Hz, 1H), 7.21 (dd, J=8.5, 2.0 Hz, 1H), 7.07-7.03 (m, 1H), 6.75 (d, J=2.0 Hz, 1H), 6.44 (d, J=2.0 Hz, 1H), 3.32 (s, 12H).

LC-MS (ESI, m/z): 646.2 [M+H]⁺.

### Example 2

### 5-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)thiophene-3-carboxamide

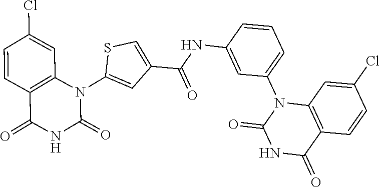 [SMILES: O=C(Nc1cccc(-n2c(=O)[nH]c(=O)c3ccc(Cl)cc32)c1)c1csc(-n2c(=O)[nH]c(=O)c3ccc(Cl)cc32)c1]

### Step 1

### 1-(3-Aminophenyl)-7-chloroquinazoline-2,4(1H,3H)-dione

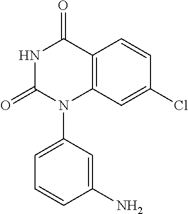 [SMILES: Nc1cccc(-n2c(=O)[nH]c(=O)c3ccc(Cl)cc32)c1]

The title compound was prepared by the procedure described in Example 1, by substituting tert-butyl (3-(7-chloro-4-(dimethylamino)-2-oxoquinazolin-1(2H)-yl)phenyl)carbamate with tert-butyl (3-(7-chloro-2,4-dioxo-3,4-dihydroquinazolin-1(2H)-yl)phenyl)carbamate in step 4. LC-MS (ESI, m/z): 288.1 [M+H]⁺.

### Step 2

### 5-(7-Chloro-2,4-dioxo-3,4-dihydroquinazolin-1(2H)-yl)thiophene-3-carboxylic acid

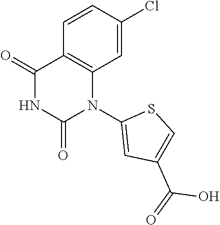 [SMILES: O=C(O)c1csc(-n2c(=O)[nH]c(=O)c3ccc(Cl)cc32)c1]

To a stirred solution of methyl 5-(7-chloro-2,4-dioxo-3,4-dihydroquinazolin-1(2H)-yl)thiophene-3-carboxylate (0.25 g, 0.7 mmol, 1.0 equiv) in 7:2:1 v/v/v THF:MeOH:water (50 mL) was added LiOH (0.09 g, 2.1 mmol, 3.0 equiv) at RT and the reaction mixture was stirred for 1 h. The reaction mixture was diluted with water (30 mL), acidified with 1 M aqueous HCl (until pH˜4) and extracted with EtOAc (2×30 mL). The combined organic layers were washed with brine (20 mL), dried over Na₂SO₄, filtered and concentrated to afford 5-(7-chloro-2,4-dioxo-3,4-dihydroquinazolin-1(2H)-yl)thiophene-3-carboxylic acid (0.2 g, 83%) as a brown solid. LC-MS (ESI, m/z): 323.1 [M+H]⁺.

### Step 3

### 5-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)thiophene-3-carboxamide

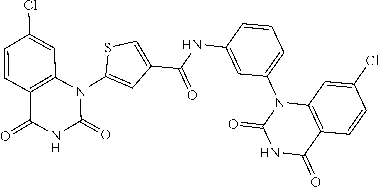 [SMILES: O=C(Nc1cccc(-n2c(=O)[nH]c(=O)c3ccc(Cl)cc32)c1)c1csc(-n2c(=O)[nH]c(=O)c3ccc(Cl)cc32)c1]

The title compound was prepared by the procedure described in Example 1, by substituting 5-(7-chloro-4-(dimethylamino)-2-oxoquinazolin-1(2H)-yl)thiophene-3-carboxylic acid with 5-(7-chloro-2,4-dioxo-3,4-dihydroquinazolin-1(2H)-yl)thiophene-3-carboxylic acid and 1-(3-aminophenyl)-7-chloro-4-(dimethylamino)quinazolin-2(1H)-one with 1-(3-aminophenyl)-7-chloroquinazoline-2,4(1H,3H)-dione in step 11. ¹HNMR (500 MHz, DMSO-d₆) δ 11.82 (br s, 2H), 10.44 (s, 1H), 8.53 (s, 1H), 8.06-8.02 (m, 2H), 7.91 (d, J=8.0 Hz, 1H), 7.85 (s, 1H), 7.72 (s, 1H), 7.62 (t, J=8.0 Hz, 1H), 7.36 (d, J=8.5 Hz, 1H), 7.32 (d, J=8.5 Hz, 1H), 7.20 (d, J=8.0 Hz, 1H), 6.70 (s, 1H), 6.43 (s, 1H). LC-MS (ESI, m/z): 592.0 [M+H]⁺.

### Example 3

### 5-(7-Chloro-4-(dimethylamino)-2-oxoquinazolin-1(2H)-yl)-N-(3-(7-chloro-4-(dimethylamino)-2-oxoquinazolin-1(2H)-yl)phenyl)-4-methylthiophene-3-carboxamide

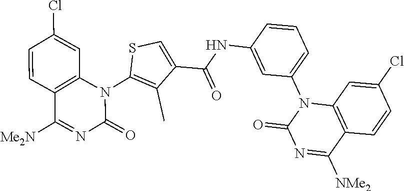 [SMILES: Cc1c(C(=O)Nc2cccc(-n3c(=O)nc(N(C)C)c4ccc(Cl)cc43)c2)csc1-n1c(=O)nc(N(C)C)c2ccc(Cl)cc21]

The title compound was prepared by the procedure described in Example 1, by substituting methyl 5-aminothiophene-3-carboxylate with methyl 5-amino-4-methylthiophene-3-carboxylate in step 7. ¹HNMR (400 MHz, DMSO-d₆) δ 10.55 (s, 1H), 8.30 (s, 1H), 8.10-8.04 (m, 2H), 7.83 (d, J=8.0 Hz, 1H), 7.75 (s, 1H), 7.58 (t, J=8.0 Hz, 1H), 7.27 (dd, J=8.4, 2.0 Hz, 1H), 7.22 (dd, J=8.4, 2.0 Hz, 1H), 7.05 (d, J=8.8 Hz, 1H), 6.57 (d, J=2.0 Hz, 1H), 6.44 (d, J=2.0 Hz, 1H), 3.33 (s, 6H), 3.31 (s, 6H), 1.98 (s, 3H). LC-MS (ESI, m/z): 660.1 [M+H]⁺.

### Example 4

### 5-(7-Chloro-4-(dimethylamino)-2-oxoquinazolin-1(2H)-yl)-N-(3-(7-chloro-4-(dimethylamino)-2-oxoquinazolin-1(2H)-yl)phenyl)-1-methyl-1H-imidazole-2-carboxamide

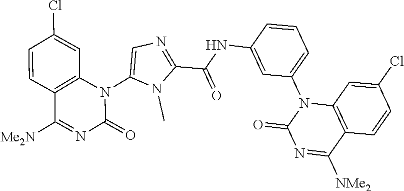 [SMILES: CN(C)c1nc(=O)n(-c2cccc(NC(=O)c3ncc(-n4c(=O)nc(N(C)C)c5ccc(Cl)cc54)n3C)c2)c2cc(Cl)ccc12]

### Step 1

### Ethyl 5-(7-chloro-4-(dimethylamino)-2-oxoquinazolin-1(2H)-yl)-1-methyl-1H-imidazole-2-carboxylate

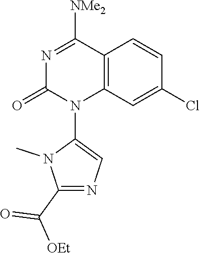 [SMILES: CCOC(=O)c1ncc(-n2c(=O)nc(N(C)C)c3ccc(Cl)cc32)n1C]

The title compound was prepared by the procedure described in Example 1, by substituting methyl 5-aminothiophene-3-carboxylate with ethyl 5-amino-1-methyl-1H-imidazole-2-carboxylate in step 7. LC-MS (ESI, m/z): 376.2 [M+H]⁺.

### Step 2

### 5-(7-Chloro-4-(dimethylamino)-2-oxoquinazolin-1(2H)-yl)-N-(3-(7-chloro-4-(dimethylamino)-2-oxoquinazolin-1(2H)-yl)phenyl)-1-methyl-1H-imidazole-2-carboxamide

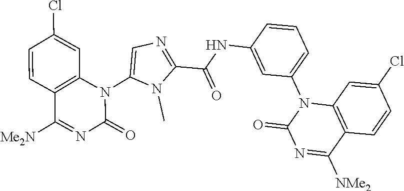 [SMILES: CN(C)c1nc(=O)n(-c2cccc(NC(=O)c3ncc(-n4c(=O)nc(N(C)C)c5ccc(Cl)cc54)n3C)c2)c2cc(Cl)ccc12]

To a stirred solution of ethyl 5-(7-chloro-4-(dimethylamino)-2-oxoquinazolin-1(2H)-yl)-1-methyl-1H-imidazole-2-carboxylate (0.10 g, 0.27 mmol, 1.00 equiv) in toluene (20 mL) was added 1-(3-aminophenyl)-7-chloro-4-(dimethylamino)quinazolin-2(1H)-one (0.087 g, 0.27 mmol, 1.00 equiv) at RT. The reaction mixture was cooled to 0° C. and Me₃Al (0.665 mL, 1.33 mmol, 5.00 equiv, 2 M in toluene) was added. The reaction mixture was stirred at 100° C. for 16 h. The reaction mixture was diluted with water (5 mL) and extracted with EtOAc (2×30 mL). The combined organic layers were washed with brine (15 mL), dried over anhydrous Na₂SO₄, filtered and concentrated. Purification by prep HPLC (mobile phase A—10 mM aqueous ammonium bicarbonate, mobile phase B—MeCN, column
X-Select C18 (19×250) mm 5mic, flow—18 mL/min, gradient method) afforded 5-(7-chloro-4-(dimethylamino)-2-oxoquinazolin-1(2H)-yl)-N-(3-(7-chloro-4-(dimethylamino)-2-oxoquinazolin-1(2H)-yl)phenyl)-1-methyl-1H-imidazole-2-carboxamide (30 mg, 17%) as an off-white solid. ¹HNMR (400 MHz, DMSO-d₆) δ 10.72 (s, 1H), 8.10-8.04 (m, 2H), 7.95 (d, J=9.2 Hz, 1H), 7.89-7.86 (m, 1H), 7.58 (t, J=8.0 Hz, 1H), 7.28 (dd, J=8.8, 2.0 Hz, 1H), 7.26 (s, 1H), 7.22 (dd, J=8.8, 2.0 Hz, 1H), 7.06 (d, J=8.0 Hz, 1H), 6.72 (d, J=2.0 Hz, 1H), 6.44 (d, J=2.0 Hz, 1H), 3.61 (s, 3H), 3.35 (s, 12H). LC-MS (ESI, m/z): 644.4 [M+H]⁺.

### Example 5

### 5-(2,4-Dioxo-7-(trifluoromethyl)-3,4-dihydroquinazolin-1(2H)-yl)-N-(3-(2,4-dioxo-7-(trifluoromethyl)-3,4-dihydroquinazolin-1(2H)-yl)phenyl)thiophene-3-carboxamide

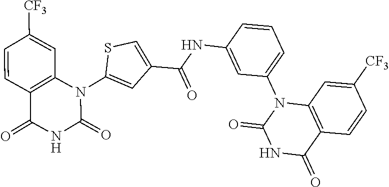 [SMILES: O=C(Nc1cccc(-n2c(=O)[nH]c(=O)c3ccc(C(F)(F)F)cc32)c1)c1csc(-n2c(=O)[nH]c(=O)c3ccc(C(F)(F)F)cc32)c1]

### Step 1

### tert-Butyl(3-(3-(2-fluoro-4-(trifluoromethyl)benzoyl)ureido)phenyl)carbamate

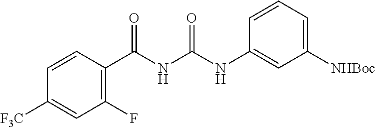 [SMILES: CC(C)(C)OC(=O)Nc1cccc(NC(=O)NC(=O)c2ccc(C(F)(F)F)cc2F)c1]

The title compound was prepared by following the same procedure described in Example 1, by replacing 4-chloro-2-fluorobenzamide with 2-fluoro-4-(trifluoromethyl)benzamide in step 1. LC-MS (ESI, m/z): 442.2 [M+H]⁺.

### Step 2

### tert-Butyl (3-(2,4-dioxo-7-(trifluoromethyl)-3,4-dihydroquinazolin-1(2H)-yl)phenyl)carbamate

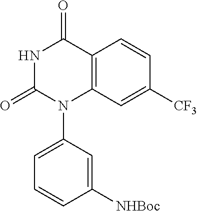 [SMILES: CC(C)(C)OC(=O)Nc1cccc(-n2c(=O)[nH]c(=O)c3ccc(C(F)(F)F)cc32)c1]

To a stirred solution of tert-butyl (3-(3-(4-chloro-2-fluorobenzoyl)ureido)phenyl)carbamate (2.5 g, 5.67 mmol, 1.00 equiv) in DMF (100 mL) was added NaH (0.408 g, 17 mmol, 3.00 equiv) at 0° C. and the reaction was stirred at rt for 1 h. The reaction mixture was quenched with ice cold water (200 ml) and then neutralized with 1 N HCl (50 ml) solid was thrown out filtered and then washed with hexane (100 mL) dried under vacuum to get tert-butyl (3-(2,4-dioxo-7-(trifluoromethyl)-3,4-dihydroquinazolin-1(2H)-yl)phenyl)carbamate (2.0 g, 83%) as an off-white solid. LC-MS (ESI, m/z): 420.2 [M−H]⁻.

### Step 3

### 1-(3-Aminophenyl)-7-(trifluoromethyl)quinazoline-2,4(1H,3H)-dione

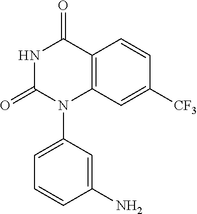 [SMILES: Nc1cccc(-n2c(=O)[nH]c(=O)c3ccc(C(F)(F)F)cc32)c1]

The title compound was prepared by following the same procedure described in Example 1, by replacing tert-butyl (3-(7-chloro-4-(dimethylamino)-2-oxoquinazolin-1(2H)-yl)phenyl)carbamate with tert-butyl (3-(2,4-dioxo-7-(trifluoromethyl)-3,4-dihydroquinazolin-1(2H)-yl)phenyl)carbamate in step 4. LC-MS (ESI, m/z): 322.1 [M+H]⁺.

### Step 4

### Methyl 5-(3-(2-fluoro-4-(trifluoromethyl)benzoyl)ureido)thiophene-3-carboxylate

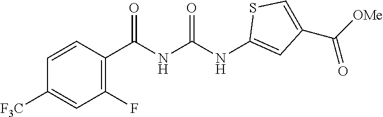 [SMILES: COC(=O)c1csc(NC(=O)NC(=O)c2ccc(C(F)(F)F)cc2F)c1]

The title compound was prepared by following the procedure described in Example 1, by substituting 4-chloro-2-fluorobenzamide with 2-fluoro-4-(trifluoromethyl)benzamide and tert-butyl (3-aminophenyl)carbamate with methyl 5-aminothiophene-3-carboxylate in step 1. LC-MS (ESI, m/z): 391.1 [M+H]⁺.

### Step 5

### Methyl 5-(2,4-dioxo-7-(trifluoromethyl)-3,4-dihydroquinazolin-1(2H)-yl)thiophene-3-carboxylate

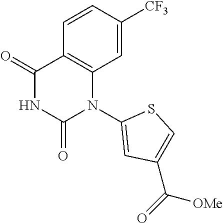 [SMILES: COC(=O)c1csc(-n2c(=O)[nH]c(=O)c3ccc(C(F)(F)F)cc32)c1]

The title compound was prepared by following the procedure described in Example 1, by replacing methyl 5-(3-(4-chloro-2-fluorobenzoyl)ureido)thiophene-3-carboxylate with methyl 5-(3-(2-fluoro-4-(trifluoromethyl)benzoyl)ureido)thiophene-3-carboxylate in step 8.

LC-MS (ESI, m/z): 371.1 [M+H]⁺.

### Step 6

### 5-(2,4-Dioxo-7-(trifluoromethyl)-3,4-dihydroquinazolin-1(2H)-yl)-N-(3-(2,4-dioxo-7-(trifluoromethyl)-3,4-dihydroquinazolin-1(2H)-yl)phenyl)thiophene-3-carboxamide

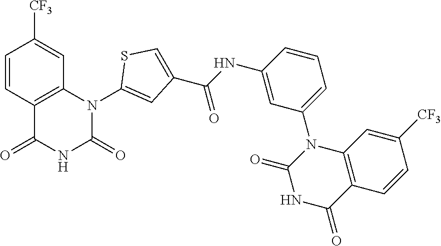 [SMILES: O=C(Nc1cccc(-n2c(=O)[nH]c(=O)c3ccc(C(F)(F)F)cc32)c1)c1csc(-n2c(=O)[nH]c(=O)c3ccc(C(F)(F)F)cc32)c1]

The title compound was prepared by following the procedure described in Example 4, by replacing 5-(7-chloro-4-(dimethylamino)-2-oxoquinazolin-1(2H)-yl)-1-methyl-1H-imidazole-2-carboxylate with methyl 5-(2,4-dioxo-7-(trifluoromethyl)-3,4-dihydroquinazolin-1(2H)-yl)thiophene-3-carboxylate and 1-(3-aminophenyl)-7-chloro-4-(dimethylamino)quinazolin-2(1H)-one with 1-(3-aminophenyl)-7-(trifluoromethyl)quinazoline-2,4(1H,3H)-dione in step 2. ¹HNMR (400 MHz, DMSO-d₆) δ 12.2-11.9 (br m, 2H), 10.49 (s, 1H), 8.55 (s, 1H), 8.27-8.21 (m, 2H), 7.94-7.87 (m, 2H), 7.73 (s, 1H), 7.67-7.57 (m, 3H), 7.22 (d, J=8.0 Hz, 1H), 6.89 (s, 1H), 6.66 (s, 1H). LC-MS (ESI, m/z): 660.1 [M+H]⁺.

### Example 6

### 5-(4-(Dimethylamino)-2-oxo-7-(trifluoromethyl)quinazolin-1(2H)-yl)-N-(3-(4-(dimethylamino)-2-oxo-7-(trifluoromethyl)quinazolin-1(2H)-yl)phenyl)thiophene-3-carboxamide

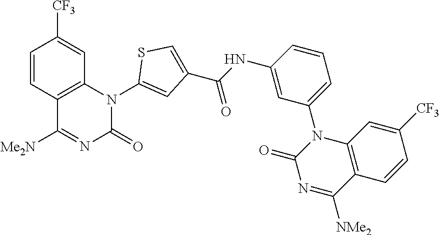 [SMILES: CN(C)c1nc(=O)n(-c2cccc(NC(=O)c3csc(-n4c(=O)nc(N(C)C)c5ccc(C(F)(F)F)cc54)c3)c2)c2cc(C(F)(F)F)ccc12]

### Step 1: NMe₂

### tert-butyl (3-(4-(dimethylamino)-2-oxo-7-(trifluoromethyl)quinazolin-1(2H)-yl)phenyl)carbamate

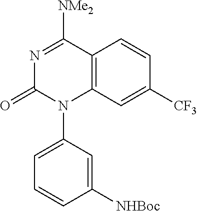 [SMILES: CN(C)c1nc(=O)n(-c2cccc(NC(=O)OC(C)(C)C)c2)c2cc(C(F)(F)F)ccc12]

The title compound was prepared by following the procedure described in Example 1, by replacing tert-butyl (3-(7-chloro-2,4-dioxo-3,4-dihydroquinazolin-1(2H)-yl)phenyl)carbamate with tert-butyl (3-(2,4-dioxo-7-(trifluoromethyl)-3,4-dihydroquinazolin-1(2H)-yl)phenyl)carbamate (synthesis described in Example 5) in step 3. LC-MS (ESI, m/z): 449.2 [M+H]⁺.

### Step 2

### 1-(3-Aminophenyl)-4-(dimethylamino)-7-(trifluoromethyl)quinazolin-2(1H)-one

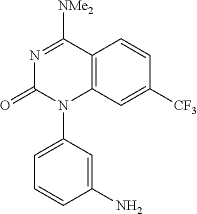 [SMILES: CN(C)c1nc(=O)n(-c2cccc(N)c2)c2cc(C(F)(F)F)ccc12]

The title compound was prepared by following the procedure described in Example 1, by replacing tert-butyl (3-(7-chloro-4-(dimethylamino)-2-oxoquinazolin-1(2H)-yl)phenyl)carbamate with tert-butyl (3-(4-(dimethylamino)-2-oxo-7-(trifluoromethyl)quinazolin-1(2H)-yl)phenyl)carbamate in step 4. LC-MS (ESI, m/z): 349.2 [M+H]⁺.

### Step 3

### Methyl 5-(4-(dimethylamino)-2-oxo-7-(trifluoromethyl)quinazolin-1(2H)-yl)thiophene-3-carboxylate

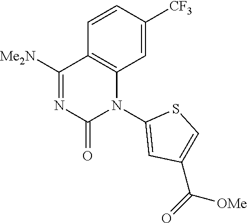 [SMILES: COC(=O)c1csc(-n2c(=O)nc(N(C)C)c3ccc(C(F)(F)F)cc32)c1]

The title compound was prepared by following the procedure described in Example 1, by replacing methyl 5-(7-chloro-4-hydroxy-2-oxoquinazolin-1(2H)-yl)thiophene-3-carboxylate with methyl 5-(2,4-dioxo-7-(trifluoromethyl)-3,4-dihydroquinazolin-1(2H)-yl)thiophene-3-carboxylate (synthesis described in Example 5) in step 9. LC-MS (ESI, m/z): 398.2 [M+H]⁺.

### Step 4

### 5-(4-(Dimethylamino)-2-oxo-7-(trifluoromethyl)quinazolin-1(2H)-yl)-N-(3-(4-(dimethylamino)-2-oxo-7-(trifluoromethyl)quinazolin-1(2H)-yl)phenyl)thiophene-3-carboxamide

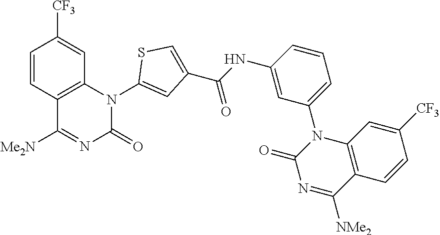 [SMILES: CN(C)c1nc(=O)n(-c2cccc(NC(=O)c3csc(-n4c(=O)nc(N(C)C)c5ccc(C(F)(F)F)cc54)c3)c2)c2cc(C(F)(F)F)ccc12]

The title compound was prepared by following the procedure described in Example 4, by replacing 5-(7-chloro-4-(dimethylamino)-2-oxoquinazolin-1(2H)-yl)-1-methyl-1H-imidazole-2-carboxylate with methyl 5-(4-(dimethylamino)-2-oxo-7-(trifluoromethyl)quinazolin-1(2H)-yl)thiophene-3-carboxylate and 1-(3-aminophenyl)-7-chloro-4-(dimethylamino)quinazolin-2(1H)-one with 1-(3-aminophenyl)-4-(dimethylamino)-7-(trifluoromethyl)quinazolin-2(1H)-one in step 2. ¹HNMR (400 MHz, DMSO-d₆) δ 10.39 (s, 1H), 8.50 (d, J=1.6 Hz, 1H), 8.28-8.23 (m, 2H), 7.88 (d, J=8.6 Hz, 1H), 7.77-7.74 (m, 1H), 7.64-7.60 (m, 2H), 7.53 (dd, J=8.4, 1.2 Hz, 1H), 7.47 (dd, J=8.4, 1.2 Hz, 1H), 7.08 (dd, J=8.8, 1.2 Hz, 1H), 6.97 (s, 1H), 6.69 (s, 1H), 3.36 (s, 6H), 3.35 (s, 6H). LC-MS (ESI, m/z): 714.2 [M+H]⁺.

### Example 7

### 3-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)benzamide

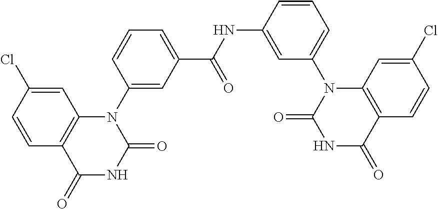 [SMILES: O=C(Nc1cccc(-n2c(=O)[nH]c(=O)c3ccc(Cl)cc32)c1)c1cccc(-n2c(=O)[nH]c(=O)c3ccc(Cl)cc32)c1]

### Step 1

### Methyl 3-(3-(4-chloro-2-fluorobenzoyl)ureido)benzoate

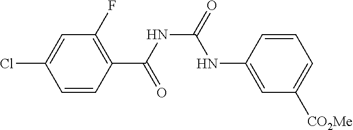 [SMILES: COC(=O)c1cccc(NC(=O)NC(=O)c2ccc(Cl)cc2F)c1]

The title compound was prepared by following the procedure described in Example 1, by substituting tert-butyl (3-aminophenyl)carbamate with methyl 3-aminobenzoate in step 1. LC-MS (ESI, m/z): 351 [M+H]⁺.

### Step 2

### Methyl 3-(7-chloro-2,4-dioxo-3,4-dihydroquinazolin-1(2H)-yl)benzoate

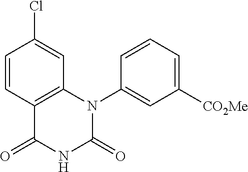 [SMILES: COC(=O)c1cccc(-n2c(=O)[nH]c(=O)c3ccc(Cl)cc32)c1]

To a stirred solution of methyl 3-(3-(4-chloro-2-fluorobenzoyl)ureido)benzoate (3.0 g, 8.57 mmol, 1.00 equiv) in THF (60 mL) was added NaH (1.03 g, 42.8 mmol, 5.00 equiv, 60% in mineral oil) at 0° C. Then reaction mixture was heated at 70° C. for 16 h. The reaction mixture was poured into ice-cold water (100 mL) and slowly added 1 N aqueous HCl (25 mL) dropwise at which time the product was precipitated as a solid. The solid was filtered and dried under vacuum to afford methyl 3-(7-chloro-2,4-dioxo-3,4-dihydroquinazolin-1(2H)-yl)benzoate (1.5 g, 55%) as an off-white solid. LC-MS (ESI, m/z): 331 [M+H]⁺.

### Step 3

### 3-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)benzamide

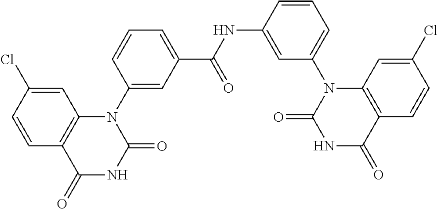 [SMILES: O=C(Nc1cccc(-n2c(=O)[nH]c(=O)c3ccc(Cl)cc32)c1)c1cccc(-n2c(=O)[nH]c(=O)c3ccc(Cl)cc32)c1]

The title compound was prepared by following the procedure described in Example 4, by replacing 5-(7-chloro-4-(dimethylamino)-2-oxoquinazolin-1(2H)-yl)-1-methyl-1H-imidazole-2-carboxylate with methyl 3-(7-chloro-2,4-dioxo-3,4-dihydroquinazolin-1(2H)-yl)benzoate and 1-(3-aminophenyl)-7-chloro-4-(dimethylamino)quinazolin-2(1H)-one with 1-(3-aminophenyl)-7-chloroquinazoline-2,4(1H,3H)-dione (synthesis described in Example 2) in step 2. ¹HNMR (400 MHz, DMSO-d₆) δ 11.89 (s, 1H), 11.82 (s, 1H), 10.66 (s, 1H), 8.18 (d, J=8.0 Hz, 1H), 8.08-8.02 (m, 3H), 7.93 (d, J=8.8 Hz, 1H), 7.88 (s, 1H), 7.80 (t, J=8.0 Hz, 1H), 7.71 (d, J=8.0 Hz, 1H), 7.62 (t, J=8.0 Hz, 1H), 7.36-7.30 (m, 2H), 7.21 (d, J=7.6 Hz, 1H), 6.43 (s, 1H), 6.38 (s, 1H). LC-MS (ESI, m/z): 584.2 [M−H]—.

### Example 8

### 2-(7-Chloro-4-(dimethylamino)-2-oxoquinazolin-1(2H)-yl)-N-(3-(7-chloro-4-(dimethylamino)-2-oxoquinazolin-1(2H)-yl)phenyl)thiazole-5-carboxamide

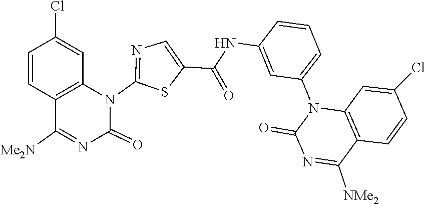 [SMILES: CN(C)c1nc(=O)n(-c2cccc(NC(=O)c3cnc(-n4c(=O)nc(N(C)C)c5ccc(Cl)cc54)s3)c2)c2cc(Cl)ccc12]

### Step 1

### Methyl 2-((4-chloro-2-fluorobenzoyl)carbamoyl)thiazole-5-carboxylate

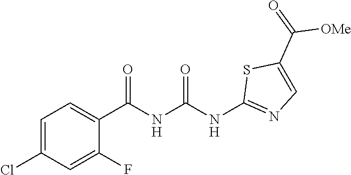 [SMILES: COC(=O)c1cnc(NC(=O)NC(=O)c2ccc(Cl)cc2F)s1]

The title compound was prepared by following the procedure described in Example 1, by substituting tert-butyl (3-aminophenyl)carbamate with methyl 2-aminothiazole-5-carboxylate in step 1. LC-MS (ESI, m/z): 358.1 [M+H]⁺.

### Step 2

### Methyl 5-(7-chloro-2,4-dioxo-3,4-dihydroquinazolin-1(2H)-yl)thiazole-2-carboxylate

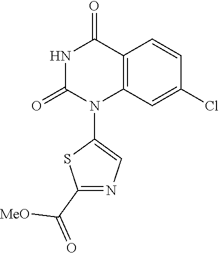 [SMILES: COC(=O)c1ncc(-n2c(=O)[nH]c(=O)c3ccc(Cl)cc32)s1]

The title compound was prepared by following the procedure described in Example 1, by substituting methyl 5-(3-(4-chloro-2-fluorobenzoyl)ureido)thiophene-3-carboxylate with Methyl 2-((4-chloro-2-fluorobenzoyl)carbamoyl)thiazole-5-carboxylate in step 8. LC-MS (ESI, m/z): 338.1 [M+H]⁺.

### Step 3

### Methyl 5-(7-chloro-4-(dimethylamino)-2-oxoquinazolin-1(2H)-yl)thiazole-2-carboxylate

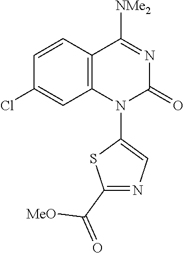 [SMILES: COC(=O)c1ncc(-n2c(=O)nc(N(C)C)c3ccc(Cl)cc32)s1]

The title compound was prepared by following the procedure described in Example 1, by replacing methyl 5-(7-chloro-4-hydroxy-2-oxoquinazolin-1(2H)-yl)thiophene-3-carboxylate with methyl 5-(7-chloro-2,4-dioxo-3,4-dihydroquinazolin-1(2H)-yl)thiazole-2-carboxylate in step 9. LC-MS (ESI, m/z): 365.2 [M+H]⁺.

### Step 4

### 2-(7-Chloro-4-(dimethylamino)-2-oxoquinazolin-1(2H)-yl)-N-(3-(7-chloro-4-(dimethylamino)-2-oxoquinazolin-1(2H)-yl)phenyl)thiazole-5-carboxamide

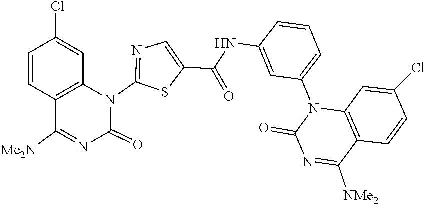 [SMILES: CN(C)c1nc(=O)n(-c2cccc(NC(=O)c3cnc(-n4c(=O)nc(N(C)C)c5ccc(Cl)cc54)s3)c2)c2cc(Cl)ccc12]

The title compound was prepared by following the procedure described in Example 4, by replacing 5-(7-chloro-4-(dimethylamino)-2-oxoquinazolin-1(2H)-yl)-1-methyl-1H-imidazole-2-carboxylate with methyl 5-(7-chloro-4-(dimethylamino)-2-oxoquinazolin-1(2H)-yl)thiazole-2-carboxylate in step 2. ¹HNMR (400 MHz, DMSO-d₆) δ 10.61 (s, 1H), 8.52 (s, 1H), 8.14 (d, J=2.0 Hz, 1H), 8.06 (d, J=8.8 Hz, 1H), 8.03 (d, J=8.8 Hz, 1H), 7.84 (d, J=8.4 Hz, 1H), 7.70 (s, 1H), 7.60 (dd, J=8.0, 8.0 Hz, 1H), 7.42 (dd, J=8.8, 2.0 Hz, 1H), 7.22 (dd, J=8.8, 2.0 Hz, 1H), 7.07 (s, 1H), 6.44 (d, J=2.0 Hz, 1H), 3.37 (s, 6H), 3.33 (s, 6H).

LC-MS (ESI, m/z): 647.1 [M+H]⁺.

### Example 9

### 5-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)-1-methyl-1H-imidazole-2-carboxamide

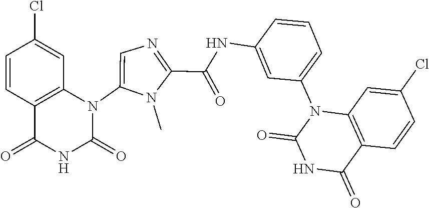 [SMILES: Cn1c(-n2c(=O)[nH]c(=O)c3ccc(Cl)cc32)cnc1C(=O)Nc1cccc(-n2c(=O)[nH]c(=O)c3ccc(Cl)cc32)c1]

### Step 1

### Ethyl 5-(7-chloro-2,4-dioxo-3,4-dihydroquinazolin-1(2H)-yl)-1-methyl-1H-imidazole-2-carboxylate

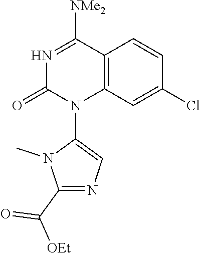 [SMILES: CCOC(=O)c1ncc(-n2c(=O)nc(N(C)C)c3ccc(Cl)cc32)n1C]

The title compound was prepared by the procedure described in Example 1, by substituting methyl 5-aminothiophene-3-carboxylate with ethyl 5-amino-1-methyl-1H-imidazole-2-carboxylate in step 7. LC-MS (ESI, m/z): 349.2 [M+H]⁺.

### Step 2

### 5-(7-Chloro-2,4-dioxo-3,4-dihydroquinazolin-1(2H)-yl)-N-(3-(7-chloro-2,4-dioxo-3,4-dihydroquinazolin-1(2H)-yl)phenyl)-1-methyl-1H-imidazole-2-carboxamide

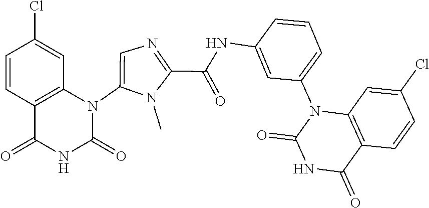 [SMILES: Cn1c(-n2c(=O)[nH]c(=O)c3ccc(Cl)cc32)cnc1C(=O)Nc1cccc(-n2c(=O)[nH]c(=O)c3ccc(Cl)cc32)c1]

The title compound was prepared by following the procedure described in Example 4, by replacing 5-(7-chloro-4-(dimethylamino)-2-oxoquinazolin-1(2H)-yl)-1-methyl-1H-imidazole-2-carboxylate with ethyl 5-(7-chloro-2,4-dioxo-3,4-dihydroquinazolin-1(2H)-yl)-1-methyl-1H-imidazole-2-carboxylate and 1-(3-aminophenyl)-7-chloro-4-(dimethylamino)quinazolin-2(1H)-one with 1-(3-aminophenyl)-7-chloroquinazoline-2,4(1H,3H)-dione (synthesis described in Example 2) in step 2. ¹HNMR (500 MHz, DMSO-d₆) S 12.07 (br s, 1H), 12.06 (s, 1H), 10.8 (s, 1H), 8.08-8.01 (m, 3H), 7.97 (t, J=8.0 Hz, 1H), 7.62 (t, J=8.0 Hz, 1H), 7.38 (d, J=8.5 Hz, 1H), 7.35 (s, 1H), 7.32 (dd, J=8.5, 1.5 Hz, 1H), 7.21 (d, J=8.0 Hz, 1H), 6.75 (d, J=5.0, 1.5 Hz, 1H), 6.43 (dd, J=7.5, 2.0 Hz, 1H), 3.72 (s, 3H). LC-MS (ESI, m/z): 590.1 [M+H]⁺.

### Example 10

### 5-(7-Bromo-4-(dimethylamino)-2-oxoquinazolin-1(2H)-yl)-N-(3-(7-chloro-4-(dimethylamino)-2-oxoquinazolin-1(2H)-yl)-2-fluorophenyl)-1-methyl-1H-pyrazole-3-carboxamide

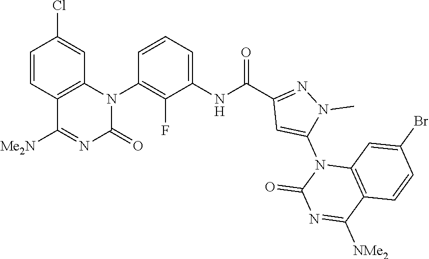 [SMILES: CN(C)c1nc(=O)n(-c2cccc(NC(=O)c3cc(-n4c(=O)nc(N(C)C)c5ccc(Br)cc54)n(C)n3)c2F)c2cc(Cl)ccc12]

### Step 1

### 4-Chloro-2-fluoro-N-((2-fluoro-3-nitrophenyl)carbamoyl)benzamide

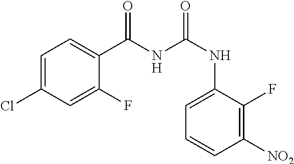 [SMILES: O=C(NC(=O)c1ccc(Cl)cc1F)Nc1cccc([N+](=O)[O-])c1F]

The title compound was prepared by following the same procedure described in Example 1, by replacing tert-butyl (3-aminophenyl)carbamate with 2-fluoro-3-nitroaniline in step 1. LC-MS (ESI, m/z): 356.2 [M+H]⁺.

### Step 2

### N-((3-Amino-2-fluorophenyl)carbamoyl)-4-chloro-2-fluorobenzamide

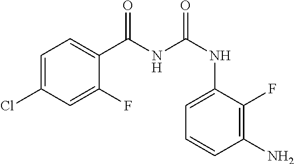 [SMILES: Nc1cccc(NC(=O)NC(=O)c2ccc(Cl)cc2F)c1F]

To a solution of the 4-chloro-2-fluoro-N-((2-fluoro-3-nitrophenyl)carbamoyl)benzamide (1.00 g, 2.81 mmol, 1.00 equiv) in TFA (25 mL) was added iron powder (0.77 g, 14.0 mmol, 5.00 equiv) and the reaction mixture was heated to 60° C. for 1 h. The reaction mixture was concentrated under reduced pressure and aqueous NaHCO₃ (100 mL) was added at 0° C. The resulting suspension was filter over Celite pad by washing with 10:1 v/v DCM and MeOH (100 mL) and dried over Na₂SO₄. Evaporation of the solvent under reduced pressure gave the crude product as a yellow gummy oil. The crude compound was triturated with diethyl ether and dried under vacuum to get N-((3-amino-2-fluorophenyl)carbamoyl)-4-chloro-2-fluorobenzamide (0.7 g, 76%) as an off-white solid. LC-MS (ESI, m/z): 326.1 [M+H]⁺.

### Step 3

### 1-(3-Amino-2-fluorophenyl)-7-chloroquinazoline-2,4(1H,3H)-dione

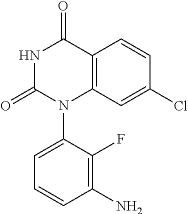 [SMILES: Nc1cccc(-n2c(=O)[nH]c(=O)c3ccc(Cl)cc32)c1F]

The title compound was prepared by the procedure described in Example 1, by replacing tert-butyl (3-(3-(4-chloro-2-fluorobenzoyl)ureido)phenyl)carbamate with N-((3-amino-2-fluorophenyl)carbamoyl)-4-chloro-2-fluorobenzamide in step 2. LC-MS (ESI, m/z): 306.1 [M+H]⁺.

### Step 4

### 1-(3-Amino-2-fluorophenyl)-7-chloro-4-(dimethylamino)quinazolin-2(1H)-one

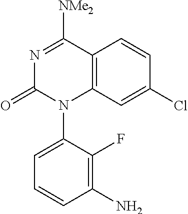 [SMILES: CN(C)c1nc(=O)n(-c2cccc(N)c2F)c2cc(Cl)ccc12]

The title compound was prepared by the procedure described in Example 1, by replacing methyl 5-(7-chloro-4-hydroxy-2-oxoquinazolin-1(2H)-yl)thiophene-3-carboxylate with 1-(3-amino-2-fluorophenyl)-7-chloroquinazoline-2,4(1H,3H)-dione in step 9. LC-MS (ESI, m/z): 333.1 [M+H]⁺.

### Step 5

### Methyl 5-(3-(4-bromo-2-fluorobenzoyl)ureido)-1-methyl-1H-pyrazole-3-carboxylate

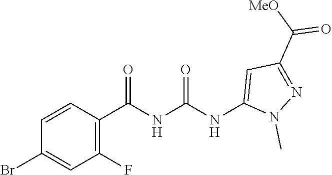 [SMILES: COC(=O)c1cc(NC(=O)NC(=O)c2ccc(Br)cc2F)n(C)n1]

The title compound was prepared by the procedure described in Example 1, by substituting tert-butyl (3-aminophenyl)carbamate with methyl 5-amino-1-methyl-1H-pyrazole-3-carboxylate and 4-chloro-2-fluorobenzamide with 4-bromo-2-fluorobenzamide in step 1. LC-MS (ESI, m/z): 399.1 [M+H]⁺.

### Step 6

### Methyl 5-(7-bromo-2,4-dioxo-3,4-dihydroquinazolin-1(2H)-yl)-1-methyl-1H-pyrazole-3-carboxylate

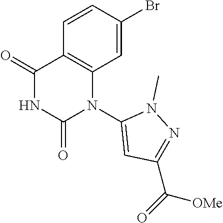 [SMILES: COC(=O)c1cc(-n2c(=O)[nH]c(=O)c3ccc(Br)cc32)n(C)n1]

The title compound was prepared by following the procedure described in Example 1, by replacing methyl 5-(3-(4-chloro-2-fluorobenzoyl)ureido)thiophene-3-carboxylate with methyl 5-(3-(4-bromo-2-fluorobenzoyl)ureido)-1-methyl-1H-pyrazole-3-carboxylate in step 8. LC-MS (ESI, m/z): 379.1 [M+H]⁺.

### Step 7

### Methyl 5-(7-bromo-4-(dimethylamino)-2-oxoquinazolin-1(2H)-yl)-1-methyl-1H-pyrazole-3-carboxylate

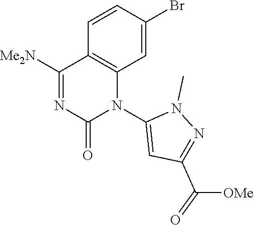 [SMILES: COC(=O)c1cc(-n2c(=O)nc(N(C)C)c3ccc(Br)cc32)n(C)n1]

The title compound was prepared by following the procedure described in Example 1, by replacing methyl 5-(7-chloro-4-hydroxy-2-oxoquinazolin-1(2H)-yl)thiophene-3-carboxylate with methyl 5-(7-bromo-2,4-dioxo-3,4-dihydroquinazolin-1(2H)-yl)-1-methyl-1H-pyrazole-3-carboxylate in step 9. LC-MS (ESI, m/z): 406.2 [M+H]⁺.

### Step 8

### 5-(7-Bromo-4-(dimethylamino)-2-oxoquinazolin-1(2H)-yl)-1-methyl-1H-pyrazole-3-carboxylic acid

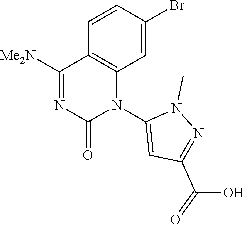 [SMILES: CN(C)c1nc(=O)n(-c2cc(C(=O)O)nn2C)c2cc(Br)ccc12]

The title compound was prepared by the procedure described in Example 1, by substituting methyl 5-(7-chloro-4-(dimethylamino)-2-oxoquinazolin-1(2H)-yl)thiophene-3-carboxylate with methyl 5-(7-bromo-4-(dimethylamino)-2-oxoquinazolin-1(2H)-yl)-1-methyl-1H-pyrazole-3-carboxylate in step 10. LC-MS (ESI, m/z): 392.1 [M+H]⁺.

### Step 9

### 5-(7-Bromo-4-(dimethylamino)-2-oxoquinazolin-1(2H)-yl)-N-(3-(7-chloro-4-(dimethylamino)-2-oxoquinazolin-1(2H)-yl)-2-fluorophenyl)-1-methyl-1H-pyrazole-3-carboxamide

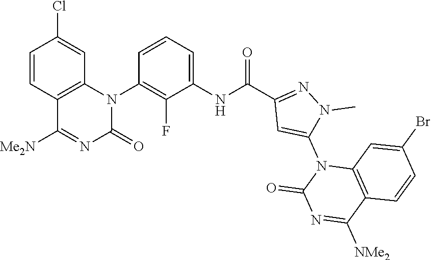 [SMILES: CN(C)c1nc(=O)n(-c2cccc(NC(=O)c3cc(-n4c(=O)nc(N(C)C)c5ccc(Br)cc54)n(C)n3)c2F)c2cc(Cl)ccc12]

To a stirred solution of methyl 5-(7-bromo-4-(dimethylamino)-2-oxoquinazolin-1(2H)-yl)-1-methyl-1H-pyrazole-3-carboxylate (0.030 g, 0.09 mmol, 1.00 equiv) in DCM (3 mL) were added methanesulfonyl chloride (0.015 g, 0.135 mmol, 1.50 equiv) and NMI (0.02 g, 0.270 mmol, 3.00 equiv) and the resultant mixture was stirred at 0° C. for 30 min. 1-(3-Amino-2-fluorophenyl)-7-chloro-4-(dimethylamino)quinazolin-2(1H)-one (0.035 g, 0.09 mmol, 1.00 equiv) was added and the mixture was stirred at RT for 16 h. The reaction mixture was diluted with water (5 mL) and extracted with EtOAc (2×10 mL). The combined organic layers were washed with brine (10 mL), dried over Na₂SO₄, filtered and concentrated.

Purification by prep-HPLC (mobile phase A—10 mM aqueous ammonium bicarbonate, mobile phase B—MeCN, column—X-Select C18 (19×250) mm 5u, flow—18 mL/min, gradient method) gave 5-(7-bromo-4-(dimethylamino)-2-oxoquinazolin-1(2H)-yl)-N-(3-(7-chloro-4-(dimethylamino)-2-oxoquinazolin-1(2H)-yl)-2-fluorophenyl)-1-methyl-1H-pyrazole-3-carboxamide as an off-white solid (10 mg, 16%). ¹HNMR (400 MHz, DMSO-d₆) δ 10.05 (s, 0.5H), 10.02 (s, 0.5H), 8.10 (d, J=8.8 Hz, 1H), 8.01 (d, J=8.8 Hz, 1H), 7.97-7.88 (m, 1H), 7.46-7.40 (m, 2H), 7.33-7.24 (m, 2H), 6.95 (s, 0.5H), 6.94 (s, 0.5H), 6.79-6.75 (m, 1H), 6.53-6.50 (m, 1H), 3.66 (s, 3H), 3.35 (s, 6H), 3.34 (s, 6H). LC-MS (ESI, m/z): 706.4 [M+H]⁺.

### Example 11

### 7-Chloro-1-(4-(((3-(7-chloro-4-(dimethylamino)-2-oxoquinazolin-1(2H)-yl)phenyl)amino)methyl)thiophen-2-yl)-4-(dimethylamino)quinazolin-2(1H)-one

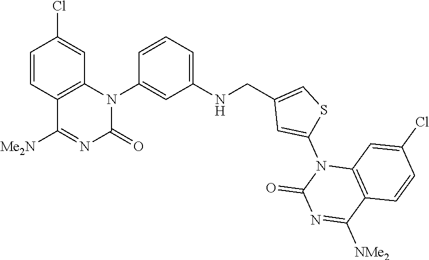 [SMILES: CN(C)c1nc(=O)n(-c2cccc(NCc3csc(-n4c(=O)nc(N(C)C)c5ccc(Cl)cc54)c3)c2)c2cc(Cl)ccc12]

### Step 1

### 7-chloro-4-(dimethylamino)-1-(4-(hydroxymethyl)thiophen-2-yl)quinazolin-2(1H)-one

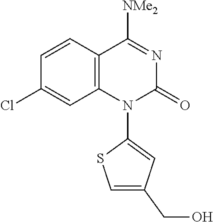 [SMILES: CN(C)c1nc(=O)n(-c2cc(CO)cs2)c2cc(Cl)ccc12]

To a stirred solution of methyl 5-(7-chloro-4-(dimethylamino)-2-oxoquinazolin-1(2H)-yl)thiophene-3-carboxylate (1.00 g, 2.75 mmol, 1.00 equiv) in DCM (20 mL) was added DIBAL-H (8.25 ml, 8.25 mmol, 1 M in hexane) at 0° C. and the resulting mixture was stirred at RT for 1 h. The reaction mixture was diluted with water (20 mL) and extracted with EtOAc (2×50 mL). The combined organic layers were washed with brine (20 mL), dried over Na₂SO₄, filtered and concentrated. To the residue was added EtOAc (20 mL) and the resulting suspension was stirred for 15 min. The solid precipitated was filtered under vacuum to afford 7-chloro-4-(dimethylamino)-1-(4-(hydroxymethyl)thiophen-2-yl)quinazolin-2(1H)-one (190 mg, 20%) as a yellow solid. LC-MS (ESI, m/z): 336.3 [M+H]⁺.

### Step 2

### 5-(7-Chloro-4-(dimethylamino)-2-oxoquinazolin-1(2H)-yl)thiophene-3-carbaldehyde

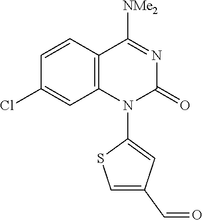 [SMILES: CN(C)c1nc(=O)n(-c2cc(C=O)cs2)c2cc(Cl)ccc12]

To a stirred solution of 7-chloro-4-(dimethylamino)-1-(4-(hydroxymethyl)thiophen-2-yl)quinazolin-2(1H)-one (80.0 mg, 0.24 mmol) in CHCl₃ (10 mL) was added MnO₂ (0.14 g, 1.43 mmol, 6.00 equiv) at 0° C. and the resulting mixture was stirred at RT for 1 h. The reaction mixture was filtered through a pad of Celite by washing with CHCl₃ (50 mL). The organic layer was washed with brine (30 mL), dried over Na₂SO₄, filtered and concentrated to afford 5-(7-chloro-4-(dimethylamino)-2-oxoquinazolin-1(2H)-yl)thiophene-3-carbaldehyde as a yellow solid (70.0 mg, 88%). LC-MS (ESI, m/z): 334.3 [M+H]⁺.

### Step 3

### 7-Chloro-1-(4-(((3-(7-chloro-4-(dimethylamino)-2-oxoquinazolin-1(2H)-yl)phenyl)amino)methyl)thiophen-2-yl)-4-(dimethylamino)quinazolin-2(1H)-one

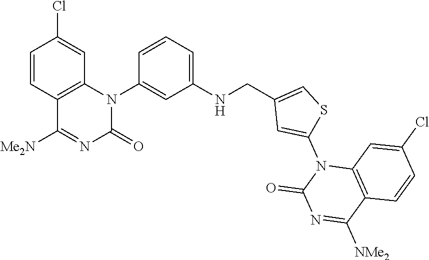 [SMILES: CN(C)c1nc(=O)n(-c2cccc(NCc3csc(-n4c(=O)nc(N(C)C)c5ccc(Cl)cc54)c3)c2)c2cc(Cl)ccc12]

To a solution of 5-(7-chloro-4-(dimethylamino)-2-oxoquinazolin-1(2H)-yl)thiophene-3-carbaldehyde (100 mg, 0.30 mmol, 1.00 equiv) in DMF (5 mL) were added 1-(3-aminophenyl)-7-chloro-4-(dimethylamino)quinazolin-2(1H)-one (90.0 mg, 0.30 mmol, 1.00 equiv) (synthesis described in Example 1) and chlorotrimethylsilane (0.08 mL, 0.60 mmol, 2.00 equiv). The resulting mixture was stirred at 0° C. for 10 min and NaBH₄ (11.3 mg, 0.30 mmol, 1.00 equiv) was added. The reaction mixture was stirred at RT for 10 min. The reaction mixture was diluted with water (10 mL) and extracted with EtOAc (2×30 mL). The combined organic layers were washed with brine (10 mL), dried over Na₂SO₄, filtered and concentrated. Purification by prep-HPLC (mobile phase A—10 mM aqueous ammonium bicarbonate, mobile phase B—MeCN, column—X-select C18 (19×250) mm 5u, flow—18 mL/min, gradient method) to afford 7-chloro-1-(4-(((3-(7-chloro-4-(dimethylamino)-2-oxoquinazolin-1(2H)-yl)phenyl)amino)methyl)thiophen-2-yl)-4-(dimethylamino)quinazolin-2(1H)-one as an off-white solid (10 mg, 5%). ¹HNMR (400 MHz, DMSO-d₆) δ 8.04-7.97 (m, 2H), 7.50 (s, 1H), 7.28 (t, J=8 Hz, 1H), 7.22 (d, J=8.8, 2.0 Hz, 1H), 7.14 (dd, J=8.4, 1.6 Hz, 1H), 7.03 (s, 1H), 6.78 (d, J=7.2 Hz, 1H), 6.63 (d, J=2.0 Hz, 1H), 6.55 (s, 1H), 6.48-6.40 (m, 3H), 4.29 (d, J=5.6 Hz, 2H), 3.30 (s, 6H), 3.29 (s, 6H). LC-MS (ESI, m/z): 632.3 [M+H]⁺.

### Example 12

### 7-Chloro-1-(4-((3-(7-chloro-4-(dimethylamino)-2-oxoquinazolin-1(2H)-yl)phenoxy)methyl)thiophen-2-yl)-4-(dimethylamino)quinazolin-2(1H)-one

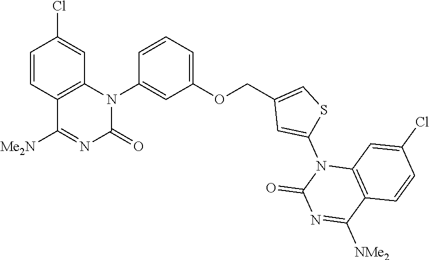 [SMILES: CN(C)c1nc(=O)n(-c2cccc(OCc3csc(-n4c(=O)nc(N(C)C)c5ccc(Cl)cc54)c3)c2)c2cc(Cl)ccc12]

### Step 1

### 3-((tert-Butyldimethylsilyl)oxy)aniline

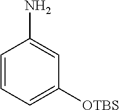 [SMILES: CC(C)(C)[Si](C)(C)Oc1cccc(N)c1]

To a stirred solution of 3-aminophenol (5.0 g, 45.8 mmol, 1.00 equiv) in DCM (50 mL) were added Et₃N (19.2 mL, 137 mmol, 3.00 equiv), DMAP (0.56 g, 4.58 mmol, 0.10 equiv) and tert-butylchlorodimethylsilane (20.7 g, 137 mmol, 3.00 equiv) and stirred at RT for 16 h. The reaction mixture was diluted with water (80 mL) and extracted with DCM (2×80 mL). The combined organic layers were washed with brine (40 mL), dried over Na₂SO₄, filtered and concentrated. Purification by silica gel chromatography afforded 3-((tert-butyldimethylsilyl)oxy)aniline (9.0 g, 87%) as a pale yellow liquid. LC-MS (ESI, m/z): 224.2 [M+H]⁺.

### Step 2

### N-((3-((tert-Butyldimethylsilyl)oxy)phenyl)carbamoyl)-4-chloro-2-fluorobenzamide

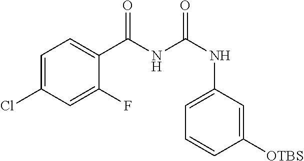 [SMILES: CC(C)(C)[Si](C)(C)Oc1cccc(NC(=O)NC(=O)c2ccc(Cl)cc2F)c1]

The title compound was prepared by following the procedure described in Example 1, by replacing tert-butyl (3-aminophenyl)carbamate with ((tert-butyldimethylsilyl)oxy)aniline in step 1. LC-MS (ESI, m/z): 423.3 [M+H]⁺.

### Step 3

### 7-Chloro-1-(3-hydroxyphenyl)quinazoline-2,4(1H,3H)-dione

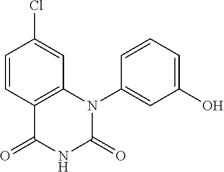 [SMILES: O=c1[nH]c(=O)n(-c2cccc(O)c2)c2cc(Cl)ccc12]

The title compound was prepared by following the procedure described in Example 1, by replacing methyl 5-(3-(4-chloro-2-fluorobenzoyl)ureido)thiophene-3-carboxylate with N-((3-((tert-butyldimethylsilyl)oxy)phenyl)carbamoyl)-4-chloro-2-fluorobenzamide in step 8.

LC-MS (ESI, m/z): 289.2 [M+H]⁺.

### Step 4

### 1-(3-((tert-Butyldimethylsilyl)oxy)phenyl)-7-chloroquinazoline-2,4(1H,3H)-dione

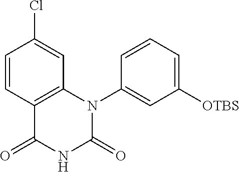 [SMILES: CC(C)(C)[Si](C)(C)Oc1cccc(-n2c(=O)[nH]c(=O)c3ccc(Cl)cc32)c1]

The title compound was prepared by following the procedure described in step 1, by replacing 3-aminophenol with 7-chloro-1-(3-hydroxyphenyl)quinazoline-2,4(1H,3H)-dione.

LC-MS (ESI, m/z): 403.3 [M+H]⁺.

### Step 5

### 1-(3-((tert-Butyldimethylsilyl)oxy)phenyl)-7-chloro-4-(dimethylamino)quinazolin-2(1H)-one

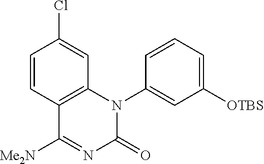 [SMILES: CN(C)c1nc(=O)n(-c2cccc(O[Si](C)(C)C(C)(C)C)c2)c2cc(Cl)ccc12]

The title compound was prepared by following the procedure described in Example 1, by replacing methyl 5-(7-chloro-4-hydroxy-2-oxoquinazolin-1(2H)-yl)thiophene-3-carboxylate with 1-(3-((tert-butyldimethylsilyl)oxy)phenyl)-7-chloroquinazoline-2,4(1H,3H)-dione in step 9. The formation of 7-chloro-4-(dimethylamino)-1-(3-hydroxyphenyl)quinazolin-2(1H)-one was also observed. LC-MS (ESI, m/z): 430.3 [M+H]⁺.

### Step 6

### 7-Chloro-4-(dimethylamino)-1-(3-hydroxyphenyl)quinazolin-2(1H)-one

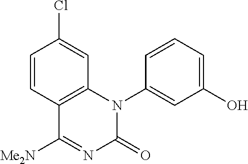 [SMILES: CN(C)c1nc(=O)n(-c2cccc(O)c2)c2cc(Cl)ccc12]

To a stirred solution of 1-(3-((tert-butyldimethylsilyl)oxy)phenyl)-7-chloro-4-(dimethylamino)quinazolin-2(1H)-one (300 mg, 0.70 mmol, 1.00 equiv) in THF (5 mL) was added TBAF (1.05 mL, 1.05 mmol, 1.50 equiv, 1 M in THF) and stirred at RT for 2 h. The reaction mixture was diluted with water (20 mL) and extracted with EtOAc (2×20 mL). The combined organic layers were washed with brine (20 mL), dried over Na₂SO₄, filtered and concentrated. Purification by silica gel chromatography afforded 7-chloro-4-(dimethylamino)-1-(3-hydroxyphenyl)quinazolin-2(1H)-one (150 mg, 68%) as an yellow solid. LC-MS (ESI, m/z): 316.3 [M+H]⁺.

### Step 7

### 7-Chloro-1-(4-(chloromethyl)thiophen-2-yl)-4-(dimethylamino)quinazolin-2(1H)-one

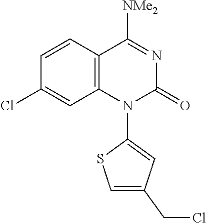 [SMILES: CN(C)c1nc(=O)n(-c2cc(CCl)cs2)c2cc(Cl)ccc12]

To a stirred solution of 7-chloro-4-(dimethylamino)-1-(4-(hydroxymethyl)thiophen-2-yl)quinazolin-2(1H)-one (120 mg, 0.36 mmol, 1.00 equiv) (synthesis described in Example 11) in DCM (5 mL) was added SOCl₂ (0.08 ml, 1.07 mmol, 3.00 equiv) at 0° C. and the reaction mixture was stirred at RT for 1 h. The reaction mixture was diluted with water (10 mL) and extracted with DCM (2×30 mL). The combined organic layers were washed with aqueous Na₂CO₃ (10 mL), brine (10 mL), dried over Na₂SO₄, filtered and concentrated to afford 7-chloro-1-(4-(chloromethyl)thiophen-2-yl)-4-(dimethylamino)quinazolin-2(1H)-one as a brown solid (90 mg, 71%). LC-MS (ESI, m/z): 354.1 [M+H]⁺.

### Step 8

### 7-Chloro-1-(4-((3-(7-chloro-4-(dimethylamino)-2-oxoquinazolin-1(2H)-yl)phenoxy)methyl)thiophen-2-yl)-4-(dimethylamino)quinazolin-2(1H)-one

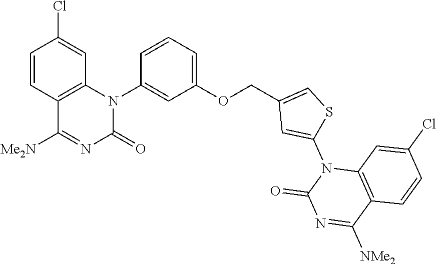 [SMILES: CN(C)c1nc(=O)n(-c2cccc(OCc3csc(-n4c(=O)nc(N(C)C)c5ccc(Cl)cc54)c3)c2)c2cc(Cl)ccc12]

To a stirred solution of 7-chloro-4-(dimethylamino)-1-(3-hydroxyphenyl)quinazolin-2(1H)-one (85.0 mg, 0.27 mmol, 1.00 equiv) in DMF (2.5 mL) was added 7-chloro-1-(4-(chloromethyl)thiophen-2-yl)-4-(dimethylamino)quinazolin-2(1H)-one (95.0 mg, 0.27 mmol, 1.00 equiv) and K₂CO₃ (55.8 mg, 0.404 mmol, 1.50 equiv) at 0° C. The reaction was stirred at RT for 36 h. The reaction mixture was diluted with water (10 mL) and extracted with EtOAc (2×20 mL). The combined organic layers were washed with brine (10 mL), dried over Na₂SO₄, filtered and concentrated. Purification by prep-HPLC (mobile phase A—0.1% aqueous formic acid, mobile phase B—MeCN, column—X-BRIDGE C18 (19×250) mm 5u, flow—18 mL/min, gradient method) afforded 7-chloro-1-(4-((3-(7-chloro-4-(dimethylamino)-2-oxoquinazolin-1(2H)-yl)phenoxy)methyl)thiophen-2-yl)-4-(dimethylamino)quinazolin-2(1H)-one (17 mg, 10%) as an off-white solid. ¹HNMR (400 MHz, DMSO-d₆) δ 8.05-8.01 (m, 2H), 7.74 (d, J=1.6 Hz, 111), 7.52 (t, J=8.4 Hz, 1H), 7.24 (dd, J=8.8, 2.0 Hz, 1H), 7.22-7.15 (m, 3H), 7.04 (t, J=2.0 Hz, 1H), 6.89 (d, J=8.0 Hz, 1H), 6.64 (d, J=2.0 Hz, 1H), 6.39 (d, J=2.0 Hz, 1H), 5.14 (s, 2H), 3.31 (s, 6H), 3.30 (s, 6H). LC-MS (ESI, m/z): 633.4 [M+H]⁺.

TABLE 1
Exemplary compounds

| EX. # | Structure |
|---|---|
| 1 | 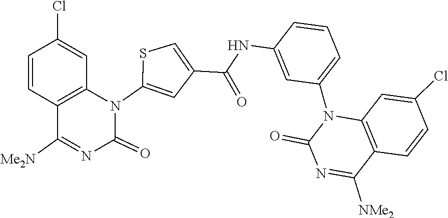 [SMILES: CN(C)c1nc(=O)n(-c2cccc(NC(=O)c3csc(-n4c(=O)nc(N(C)C)c5ccc(Cl)cc54)c3)c2)c2cc(Cl)ccc12] |
| 2 | 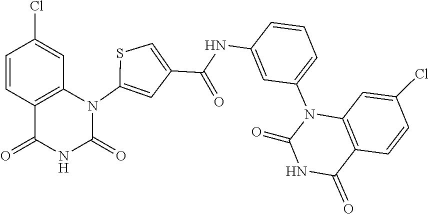 [SMILES: O=C(Nc1cccc(-n2c(=O)[nH]c(=O)c3ccc(Cl)cc32)c1)c1csc(-n2c(=O)[nH]c(=O)c3ccc(Cl)cc32)c1] |
| 3 | 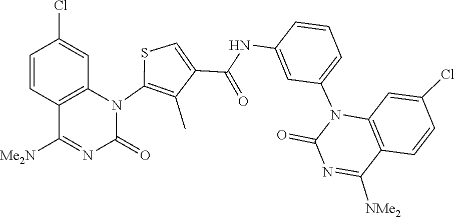 [SMILES: Cc1c(C(=O)Nc2cccc(-n3c(=O)nc(N(C)C)c4ccc(Cl)cc43)c2)csc1-n1c(=O)nc(N(C)C)c2ccc(Cl)cc21] |
| 4 | 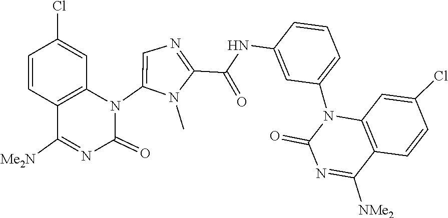 [SMILES: CN(C)c1nc(=O)n(-c2cccc(NC(=O)c3ncc(-n4c(=O)nc(N(C)C)c5ccc(Cl)cc54)n3C)c2)c2cc(Cl)ccc12] |
| 5 | 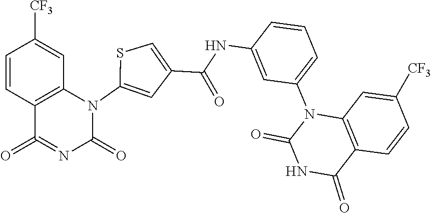 |
| 6 | 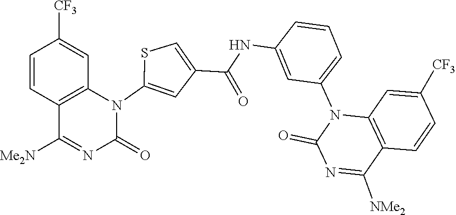 [SMILES: CN(C)c1nc(=O)n(-c2cccc(NC(=O)c3csc(-n4c(=O)nc(N(C)C)c5ccc(C(F)(F)F)cc54)c3)c2)c2cc(C(F)(F)F)ccc12] |
| 7 | 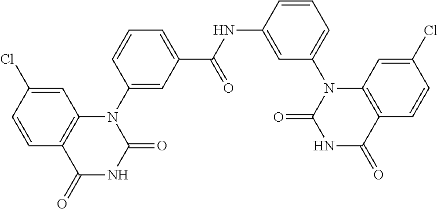 [SMILES: O=C(Nc1cccc(-n2c(=O)[nH]c(=O)c3ccc(Cl)cc32)c1)c1cccc(-n2c(=O)[nH]c(=O)c3ccc(Cl)cc32)c1] |
| 8 | 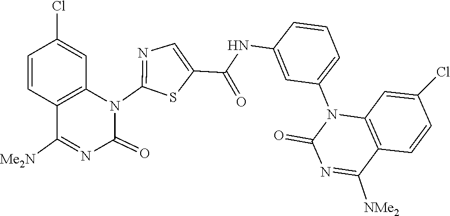 [SMILES: CN(C)c1nc(=O)n(-c2cccc(NC(=O)c3cnc(-n4c(=O)nc(N(C)C)c5ccc(Cl)cc54)s3)c2)c2cc(Cl)ccc12] |
| 9 | 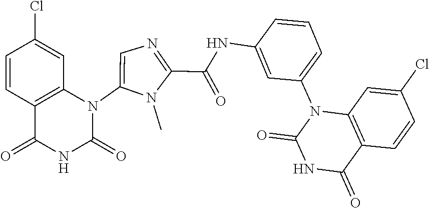 [SMILES: Cn1c(-n2c(=O)[nH]c(=O)c3ccc(Cl)cc32)cnc1C(=O)Nc1cccc(-n2c(=O)[nH]c(=O)c3ccc(Cl)cc32)c1] |
| 10 | 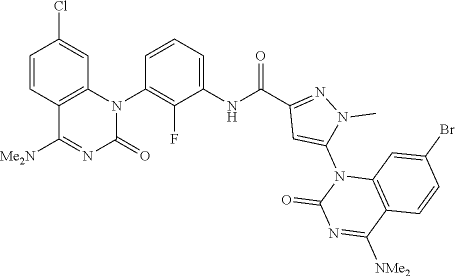 [SMILES: CN(C)c1nc(=O)n(-c2cccc(NC(=O)c3cc(-n4c(=O)nc(N(C)C)c5ccc(Br)cc54)n(C)n3)c2F)c2cc(Cl)ccc12] |
| 11 | 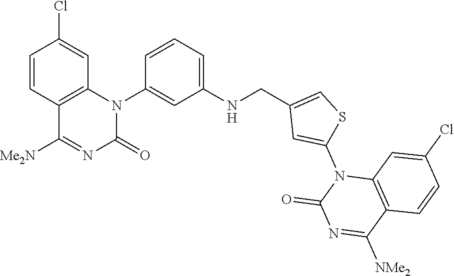 [SMILES: CN(C)c1nc(=O)n(-c2cccc(NCc3csc(-n4c(=O)nc(N(C)C)c5ccc(Cl)cc54)c3)c2)c2cc(Cl)ccc12] |
| 12 | 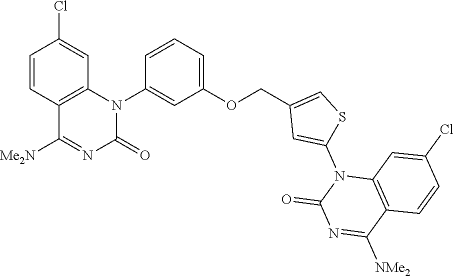 [SMILES: CN(C)c1nc(=O)n(-c2cccc(OCc3csc(-n4c(=O)nc(N(C)C)c5ccc(Cl)cc54)c3)c2)c2cc(Cl)ccc12] |

### Biological Examples

The ability of the compound of present disclosure to inhibit MAT2A enzyme was determined using a Phosphate Sensor Fluorescence Assay described below.

### Biologic Example. Phosphate Sensor Fluorescence Assay

MAT2A enzyme is incubated with a test compound in DMSO or DMSO and its substrates (L-methionine and ATP) in a microtiter plate. The enzymatic reaction is stopped by the addition of Working Phosphate Sensor Mixture. The plate is analyzed for fluorescence at 450 nm. The high control (DMSO with enzyme and its substrates) gives high fluorescence which represents no inhibition of enzymatic activity while the low control (DMSO with MAT2A substrates and no enzyme) gives low fluorescence which represents full inhibition of enzymatic activity.

### Materials:

Human MAT2A: Cepter, amino acids 1-395 (Clone ID 334, lot 00023-148)
Tris, pH 7.5: Invitrogen cat #15567-027
KCl: Ambion cat #AM9640G
MgCl2: Ambion cat #AM9530G
Brij-35: Sigma cat B4184-10ML
DTT: Goldbio cat #DTT100
BGG: Sigma cat #G5009-25G
PNP: Novus Biologicals cat #NBP1-50872
7-MEG: Cayman Chemical cat #15988
L-Methionine: JTBaker P/N 2085-05
ATP: Promega P/N V915B
Phosphate Sensor: Thermo Fisher cat #PV4407
EDTA: Invitrogen cat #15575-038
Assay plate: 384-well black polypropylene plate: Corning P/N 3573

### Final Assay Conditions:

Assay Buffer: 50 mM Tris, pH 7.5/50 mM KCl/10 mM MgCl₂/0.01% Brij-35/1 mM DTT/0.01% BGG/40 nM PNP/6 uM 7-MEG
MAT2A: 5.8 nM MAT2A (1-395), Cepter, clone ID 334, lot 00023-148 before the addition of Working Phosphate Sensor Mixture
L-methionine: 500 uM before the addition of Working Phosphate Sensor Mixture
ATP: 500 uM before the addition of Working Phosphate Sensor Mixture

### Procedure:

For the assay, a mixture of 1 mM L-methionine/1 mM ATP (2× final pre-stopped concentration) in assay buffer; MAT2A (2× final pre-stopped concentration) in Assay Buffer and Working Phosphate Sensor Mixture (1.5 uM Phosphate Sensor/30 mM EDTA in Assay Buffer, which is 3× final concentrations) were prepared. Test compounds or DMSO were added to the appropriate well using a Beckman Coulter Echo 550 acoustic liquid handler. 10 μl/well of Assay Buffer was added to the wells corresponding to the negative control and 10 μl/well of MAT2A was added to all the wells except for those corresponding to the negative control. After incubating the plate at room temperature for 15 minutes, 10 μl/well of the 1 mM L-methionine/1 mM ATP mixture was added to all wells. The plate was centrifuged at 1000 rpm for 1 minute and then incubated at room temperature for 1 hour. 10 μl of the Working Phosphate Sensor Mixture was added to all wells and the plate was centrifuged at 1000 rpm for 1 minute. The plate was read for fluorescence at 450 nm after exciting at 430 nm on a Tecan Spark microplate reader.

### Data Analysis:

Percent inhibition was calculated in Chemical and Biological Information System (CBIS), (ChemInnovation Software Inc.). Curves were fitted by CBIS as % inhibition vs. log [compound concentration] using a 4-parameter inhibition model.

$$Fit = ( A + ( ( B - A ) / ( 1 + ( ( C / x ) \hat{} D ) ) ) )$$

$$Res = ( y - fit )$$

The IC₅₀ of a representative number of compounds in Table 1 above are disclosed in Table 2 below:

(+)IC₅₀=10 μM-1 μM;(++)IC₅₀=1μM-500 nM;(+++)IC₅₀=500 nM-200 nM;(++++) IC₅₀<200 nM

TABLE 2

| Example | IC₅₀ |
|---|---|
| 1 | ++++ |
| 2 | ++++ |
| 3 | ++++ |
| 4 | ++++ |
| 5 | ++++ |
| 6 | ++++ |
| 7 | ++++ |
| 8 | ++++ |
| 9 | ++++ |
| 10 | ++++ |
| 11 | ++++ |
| 12 | ++++ |

What is claimed:

1. A compound having the Formula (I) 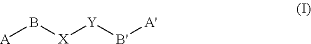
or a pharmaceutically acceptable salt thereof, wherein
A and A′ are each independently selected from the group consisting of: 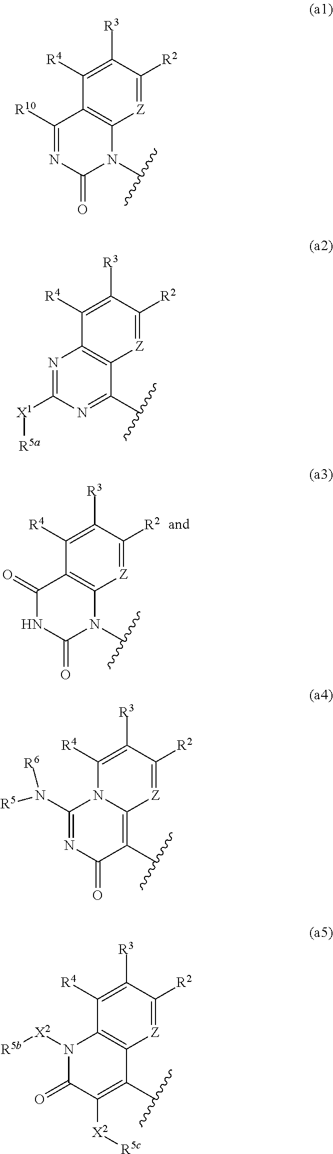
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁-6 alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}-R^{c}, —SR^{c}, —S—X^{a}-R^{c}, —NR^{f}R^{g}, —S(O)₂R^{c}, —S(O)₂—X^{a}-R^{c}, —S(O)₂-NR^{h}R^{c}, —S(O)₂—NR^{h}—X^{a}-R^{c}, —NR^{h}—S(O)₂—R^{c}, —NR^{h}—S(O)₂—X^{a}-R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{Y}, —C(O)R^{Y}, —X^{b}—C(O)R^{y}, —C(O)OR^{Y}, and -X^{b}-C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

2. The compound of claim 1, or a pharmaceutically acceptable salt thereof,
wherein
A and A′ are each independently selected from the group consisting of: 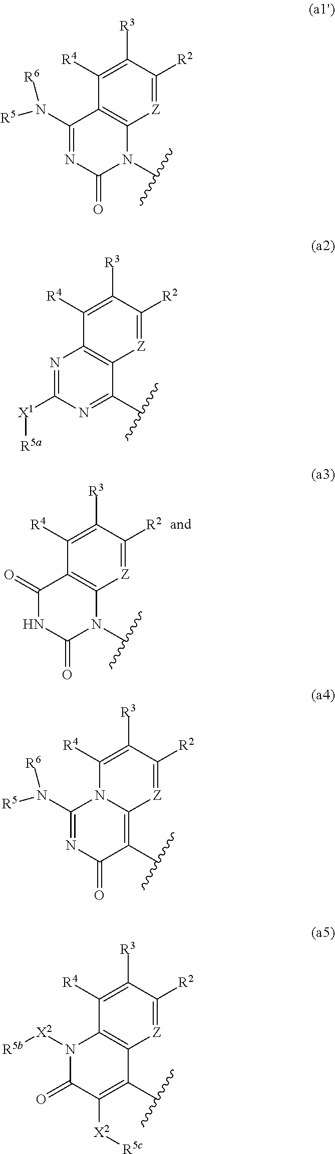
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(CH₃), O and S;
Y is selected from the group consisting of CH₂, C(O), NH, N(CH₃), O and S;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R⁵ and R⁶ are each independently selected from the group consisting of H, C₁₋₆ alkyl, C₁₋₆ haloalkyl, and C₃₋₆ cycloalkyl;
each R⁷ is independently selected from the group consisting of halogen, cyano, C₁₋₃ alkyl, and C₁₋₃ haloalkyl;
X¹ is selected from the group consisting of a bond, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond and C₁₋₄ alkylene;
R^{5c} is selected from the group consisting of H, halo, cyano, C₁₋₆ alkyl; C₁₋₆ haloalkyl, —OR^{c}, —O—X^{a}—R^{c}, —SR^{c}, —S—X^{a}—R^{c}, —NR^{f}R^{g}, —S(O)₂R^{c}, —S(O)₂—X^{a}—R^{c}, —S(O)₂—NR^{h}R^{c}, —S(O)₂—NR^{h}—X^{a}—R^{c}, —NR^{h}—S(O)₂—R^{c}, —NR^{h}—S(O)₂—X^{a}—R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{y}, —X^{b}—C(O)R^{y}, —C(O)OR^{Y}, and —X^{b}—C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

3. The compound of claim 1, or a pharmaceutically acceptable salt thereof, wherein each A and A′ has the formula:

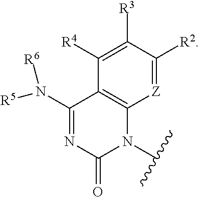

4. The compound of claim 1, or a pharmaceutically acceptable salt thereof, wherein each A and A′ has the formula:

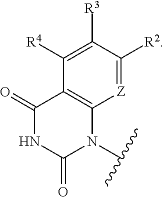

5. The compound of claim 1, or a pharmaceutically acceptable salt thereof, wherein only one of A and A′ has the formula:

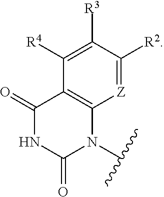

6. The compound of claim 1, or a pharmaceutically acceptable salt thereof, wherein one or both of B and B′ is phenyl, which is unsubstituted or substituted with from 1 to 3 R⁷.

7. The compound of claim 1, or a pharmaceutically acceptable salt thereof, wherein one or both of B and B′ is a 5- or 6-membered heteroaryl group having from one to three heteroatoms as ring vertices selected from N, O and S; and is unsubstituted or substituted with from 1 to 3 R⁷.

8. The compound of claim 1, or a pharmaceutically acceptable salt thereof, wherein one or both of B and B′ is selected from the group consisting of thiazolyl, imidazolyl, pyrazolyl, and thienyl; and is unsubstituted or substituted with from 1 to 3 R⁷.

9. The compound of claim 1, or a pharmaceutically acceptable salt thereof, wherein X and Y are combined to form —C(O)NH—.

10. The compound of claim 1, or a pharmaceutically acceptable salt thereof, wherein X and Y are combined to form —CH₂NH—.

11. The compound of claim 1, or a pharmaceutically acceptable salt thereof, wherein X and Y are combined to form —CH₂O—.

12. The compound of claim 1, or a pharmaceutically acceptable salt thereof, wherein each R¹, R², R³ and R⁴ are each independently selected from the group consisting of H, C₁₋₆ alkyl, C₁₋₆ haloalkyl, and halo.

13. The compound of claim 12, or a pharmaceutically acceptable salt thereof, wherein only one of R¹, R², R³ and R⁴ is other than H.

14. The compound of claim 1, or a pharmaceutically acceptable salt thereof, wherein each of R⁵ and R⁶ is methyl.

15. A pharmaceutical composition comprising a compound of claim 1, or a pharmaceutically acceptable salt thereof and at least one pharmaceutically acceptable excipient.

16. A method for treating a disease mediated by MAT2A in a patient comprising administering to the patient a therapeutically effective amount of a compound Formula (I): 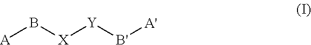
or a pharmaceutically acceptable salt thereof, wherein
A and A′ are each independently selected from the group consisting of: 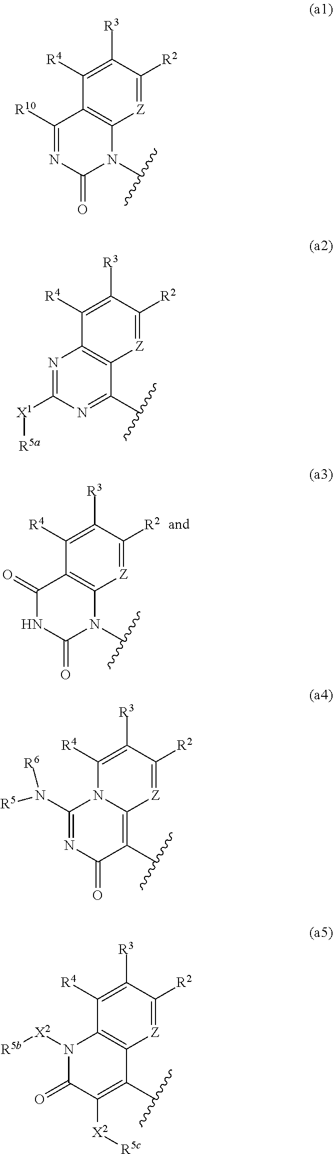
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}—R^{c}, —SR^{c}, —S—X^{a}—R^{c}, —NR^{f}R^{g}, —S(O)₂R^{c}, —S(O)₂—X^{a}—R^{c}, —S(O)₂—NR^{h}R^{c}, —S(O)₂—NR^{h}—X^{a}—R^{c}, —NR^{h}—S(O)₂—R^{c}, —NR^{h}—S(O)₂—X^{a}—R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{y}, —X^{b}—C(O)R^{y}, —C(O)OR^{Y}, and —X^{b}—C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

17. The method of claim 16, wherein the disease is cancer.

18. A method of treating a MTAP null cancer in a patient comprising administering to the patient a therapeutically effective amount of a compound of Formula (I): 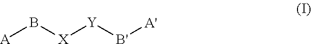
or a pharmaceutically acceptable salt thereof, wherein
A and A′ are each independently selected from the group consisting of: 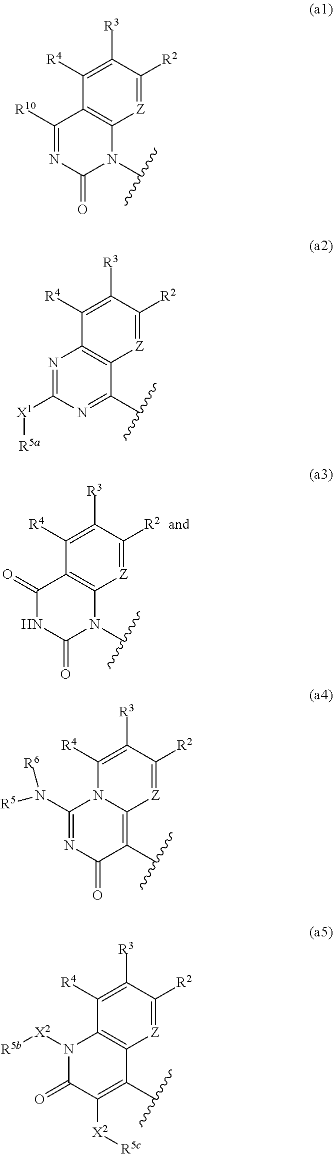
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}—R^{c}, —SR^{c}, —S—X^{a}—R^{c}, —NR^{f}R^{g}, —S(O)₂R^{c}, —S(O)₂—X^{a}—R^{c}, —S(O)₂—NR^{h}R^{c}, —S(O)₂—NR^{h}—X^{a}—R^{c}, —NR^{h}—S(O)₂—R^{c}, —NR^{h}—S(O)₂—X^{a}—R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{y}, —C(O)R^{y}, —X^{b}—C(O)R^{y}, —C(O)OR^{Y}, and —X^{b}—C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

19. A method for treating a cancer in a patient, wherein the cancer is characterized by a reduction or absence of MTAP gene expression, the absence of the MTAP gene, reduced function of MTAP protein, reduced level or absence of MTAP protein, MTA accumulation, or combination thereof, comprising administering to the subject a therapeutically effective amount of a compound of Formula (I): 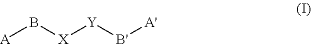
or a pharmaceutically acceptable salt thereof, wherein
A and A′ are each independently selected from the group consisting of: 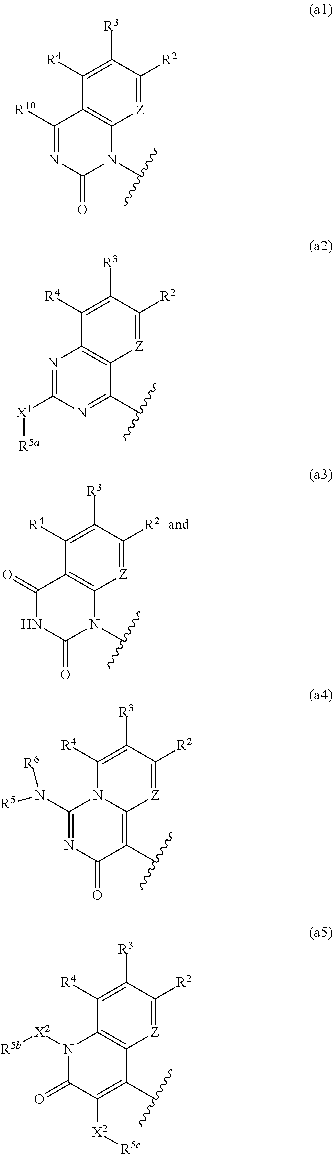
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R⁷ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}—R^{c}, —SR^{c}, —S—X^{a}—R^{c}, —NR^{f}R^{g}, —S(O)₂R^{c}, —S(O)₂—X^{a}—R^{c}, —S(O)₂—NR^{h}R^{c}, —S(O)₂—NR^{h}—X^{a}—R^{c}, —NR^{h}—S(O)₂—R^{c}, —NR^{h}—S(O)₂—X^{a}—R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{Y}, —C(O)R^{y}, —X^{b}—C(O)R^{y}, —C(O)OR^{Y}, and -X^{b}—C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

20. Use of a compound of Formula (I) or a pharmaceutically acceptable salt thereof in the manufacture of a medicament for the treatment of cancer, wherein the compound of Formula (I) is: 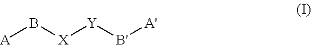
or a pharmaceutically acceptable salt thereof, wherein
A and A′ are each independently selected from the group consisting of: 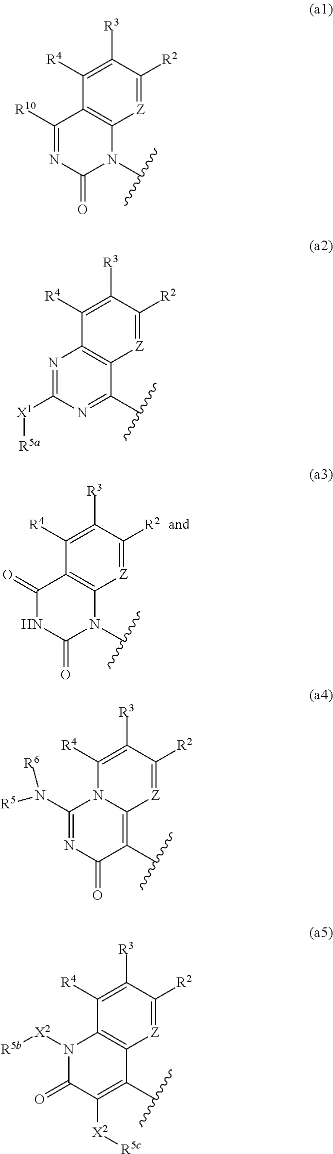
wherein the wavy line indicated the point of attachment to B or B′;
B and B′ are each independently selected from the group consisting of phenyl and a 5- or 6-membered heteroaryl group having from one to three heteroatoms as ring vertices selected from N, O and S; and wherein each B and B′ is unsubstituted or substituted with from 1 to 3 R⁷;
X is selected from the group consisting of CH₂, C(O), NH, N(R⁸), O and S;
wherein R⁸ is C₁₋₆ alkyl, C₁₋₆alkoxyC₁₋₆alkyl, or C₃₋₆ cycloalkyl;
Y is selected from the group consisting of CH₂, C(O), NH, N(R⁹), O and S;
wherein R^{g} is C₁₋₆ alkyl, C₁₋₆alkoxyC₁₋₆alkyl, or C₃₋₆ cycloalkyl;
and wherein X and Y are not both C(O), and at least one of X and Y is CH₂ or C(O);
Z is C(R¹) or N;
each R¹, R², R³ and R⁴ are each independently selected from the group consisting of H, C₁₋₆ alkyl, C₁₋₆ haloalkyl, C₁₋₆ alkoxy, cyano, halo and C₃₋₈ cycloalkyl, wherein the cycloalkyl group is substituted with from 0 to 2 groups independently selected from the group consisting of C₁₋₄ alkyl and halo;
R¹⁰ is —NR⁵R⁶ or —OR¹¹;
R⁵ and R⁶ are each independently selected from the group consisting of H, C₁₋₆ alkyl, C₁₋₆ haloalkyl, hydroxyC₁₋₆alkyl, C₁₋₆alkoxyC₁₋₆alkyl, and C₃₋₆ cycloalkyl; or
R⁵ and R⁶ combine to form 3-7 membered heterocycloalkyl having 1 to 3 heteroatom ring vertices independently selected from the group consisting of N, O, and S;
R¹¹ is C₁₋₆ alkyl;
each R¹ is independently selected from the group consisting of halogen, cyano, C₁₋₃ alkyl, and C₁₋₃ haloalkyl;
X¹ is selected from the group consisting of a bond, O, N(R¹²), S, C₁₋₄ alkylene, and phenylene;
R^{5a} is selected from the group consisting of H, halo, cyano, —NR^{a}R^{b}, —OR^{c}, —SR^{c}, —C(O)R^{d}, —C(O)OR^{d}, —C(O)NR^{a}R^{b} and a 6- to 10-membered heteroaryl ring having 1 to 3 heteroatom ring vertices independently selected from the group consisting of N, O, and S;
X² is selected from the group consisting of a bond, O, and C₁₋₄ alkylene;
R^{5b} is selected from the group consisting of H, halo, C₁₋₆ alkyl, C₁₋₆ haloalkyl, C₆₋₁₀ aryl, —NR^{a}R^{b}, —C(O)R^{d}, —C(O)OR^{d}, —C(O)NR^{a}R^{b}, and a 6- to 10-membered heteroaryl ring having 1 to 3 heteroatom ring vertices independently selected from the group consisting of N, O, and S;
X³ is selected from the group consisting of a bond, O, N(R¹²), S, and C₁₋₄ alkylene;
each R¹² is independently H or C₁₋₆ alkyl;
R^{5c} is selected from the group consisting of H, halo, cyano, C₁₋₆ alkyl; C₁₋₆ haloalkyl, —OR^{c}, —O—X^{a}—R^{c}, —SR^{c}, —S—X^{a}—R^{c}, —NR^{f}R^{g}, —S(O)₂R^{c}, —S(O)₂—X^{a}—R^{c}, —S(O)₂—NR^{h}R^{c}, —S(O)₂—NR^{h}—X^{a}—R^{c}, —NR^{h}—S(O)₂—R^{c}, —NR^{h}—S(O)₂—X^{a}—R^{c}, and a 3- to 6-membered heterocycloalkyl having 1 to 3 heteroatom ring vertices independently selected from the group consisting of N, O, and S;
each R^{a} and R^{b} is independently selected from the group consisting of H, C₁₋₆ alkyl, C₁₋₆ haloalkyl, and phenyl, wherein the phenyl is substituted with 0 to 2 moieties independently selected from the group consisting of C₁₋₄ alkyl, —OR^{x}, and —X^{a}—OR^{x};
each R^{c} is independently selected from the group consisting of H, C₁₋₆ alkyl, C₁₋₆ haloalkyl, C₃₋₆ cycloalkyl, and a 3- to 6-membered heterocycloalkyl having 1 to 3 heteroatom ring vertices independently selected from the group consisting of N, O, and S, wherein the cycloalkyl and the 3- to 6-membered heterocycloalkyl are each independently substituted with 0 to 2 moieties independently selected from the group consisting of C₁₋₄ alkyl, —OR^{y}, —X^{b}—OR^{Y}, —C(O)R^{Y}, —X^{b}—C(O)R^{y}, —C(O)OR^{y}, and —X^{b}—C(O)OR^{y};
R^{d} is selected from the group consisting of H, C₁₋₆ alkyl, and C₁₋₆ haloalkyl;
R^{f} and R^{g} are each independently selected from the group consisting of H, C₁₋₆ alkyl, C₁₋₆ haloalkyl, and phenyl; or R^{f} and R^{g} together with the nitrogen to which they are attached combine to form a 4- to 6-membered heterocycloalkyl ring comprising 0 to 2 additional heteroatoms independently selected from the group consisting of N, O, and S, wherein the phenyl and the 4- to 6-membered heterocycloalkyl are substituted with 0 to 2 moieties independently selected from the group consisting of C₁₋₄ alkyl, —OR^{x}, and —X^{a}—OR^{x};
R^{h} is selected from the group consisting of H, C₁₋₆ alkyl, C₁₋₆ haloalkyl;
each R^{x} and R^{y} is selected from the group consisting of H, C₁₋₄ alkyl, and C₁₋₄ haloalkyl; and
each X^{a} and X^{b} is independently C₁₋₃ alkylene.

21. The method or use of any one of claims 17 to 20, wherein the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophagogastric cancer, malignant peripheral nerve sheath tumor, leiomyosarcoma, cholangiocarcinoma, and mesothelioma.
